(19) United States
(12) Patent Application Publication
(10) Pub. No.: US 2008/0044844 A1
(43) Pub. Date: Feb. 21, 2008

(54) Populations Of Cells And Devices And Systems Including Same

(76) Inventors: Shimshon Belkin, Kiryat Ono (IL); Rami Pedahzur, Yehuda (IL); Rachel Rosen, ModiIn (IL); Itai Benovici, Jerusalem (IL); Yosi Shacham-Diamand, Zikhron-Yaakov (IL); Arthur Rabner, Natania (IL); Mark Oksman, Bat-yam (IL)

Correspondence Address: Martin D. Moynihan;PRTSI, P.O.Box 16446, Arlington, VA, 22215, US

(21) Appl. No.: 10/587,515

(22) PCT Filed: Jan. 25, 2005
(86) PCT No.: PCT/IL05/00091

§ 371 Date: Jun. 6, 2007

Related U.S. Application Data
(60) Provisional application No. 60/539,097, filed on Jan. 27, 2004.
(60) Provisional application No. 60/565,815, filed on Apr. 28, 2004.

(51) Int. Cl. C12Q 1/68; C12M 1/00; C12N 5/06

(52) U.S. Cl. 435/29; 435/287.1; 435/325

(57) ABSTRACT

A population of cells is provided. The population of cells comprising at least two subpopulations of cells, wherein a first subpopulation of the at least two subpopulation of cells includes a first reporter expression construct being expressible in a cell of the first subpopulation when exposed to a first analyte and whereas a second subpopulation of the at least two subpopulation of cells includes a second reporter expression construct being expressible in a cell of the second subpopulation when exposed to a second analyte.

### FIELD AND BACKGROUND OF THE INVENTION

The present invention relates to novel populations of cells and, more particularly, to devices and systems which utilize such cell populations for analyte detection.

The ability to qualify and quantify substances present in a liquid or gaseous samples is of great importance in clinical, environmental, health and safety, remote sensing, military, food/beverage and chemical processing applications.

There are two general approaches for analyte (i.e., substance) detection. Traditional approaches are based on chemical or physical analysis allowing highly accurate and sensitive determination of the exact composition of any sample [e.g., liquid chromatography (LC), gas chromatography (GC), and supercritical fluid chromatography.(SFC)]. However, these techniques are time consuming, extremely expensive, require sample preconcentration, and are difficult or impossible to adapt to field use. In addition, such technologies fail to provide data as to the bioavailability of pollutants, their effects on living systems, and their synergistic/antagonistic behavior in mixtures.

A biosensor is a device that qualifies and/or quantifies a physiological or biochemical signal. Biosensors have been developed to overcome some of the shortcomings of the classical analyte detection techniques. Good biosensing systems are characterized by specificity, sensitivity, reliability, portability, ability to function even in optically opaque solutions, real-time analysis and simplicity of operation. Biosensors couple a biological component with an electronic transducer and thus enable conversion of a biochemical signal into a quantifiable electrical response.

The function of the biosensor depends on the biochemical specificity of the biologically active material. Enzymes, antibodies, aptamers, DNA, receptors, organelles and microorganisms as well as plant cells or tissues have been used as biological sensing elements. The most commonly used biological element in the construction of biosensors are enzymes, due to their high specific activities as well as high analytical specificity. Purified enzymes are, however, expensive and unstable, thus limiting their applications in the field of biosensors.

The use of whole cells as the biosensing element negates the lengthy procedure of enzyme purifications, preserves the enzymes in their natural environment and protects it from inactivation by external toxicants such as heavy metals. Whole cells also provide a multipurpose catalyst especially when the process requires the participation of a number of enzymes in sequence. Whole cells have been used either in viable or non-viable form. Viable microbes, for example, can metabolize various organic compounds resulting in various end products like ammonia, carbon dioxide, acids and the like, which can be monitored using a variety of transducers [Burlage (1994) Annu. Rev. Microbiol. 48: 291-309; Riedel (1998) Anal. Lett. 31:1-12; Arikawa (1998) Mulchandani, Rogers (Eds.) Enzyme and Microbial Biosensors: Techniques and Protocols. Humanae Press, Totowa, N.J., pp. 225-235; and Simonian (1998) Mulchandani, Rogers (Eds.) Enzyme and Microbial Biosensors: Techniques and Protocols. Humanae Press, Totowa, N.J. pp: 237-248].

The selection of microbial culture which corresponds well with a spectrum of compounds present in the sample is of significant importance.

A number of selection approaches are known, in the art. For example, adaptation of a microbe for induction of desirable metabolic pathways and uptake systems can be effected by cultivation in a medium containing appropriate substrates [Di Paolantonio and Rechnitz (1982) supra; Riedel (1990) Anal. Lett. 23:757-770; Fleschin (1998) Prep. Biochem. Biotechnol. 28:261-269]. Specifically, for the biochemical degradation of complex substrates such as mixtures of phenols, the use of activated sludge obtained from waste treatment plants can serve as an acclimatized mixed microbial consortium as compared to pure cultures [Joshi and D'souza (1999) J. Environ. Sci. Health Part A Eviron. Sci. Engng. 34:1689-1700].

Alternatively, when a single cell does not contain all enzymes necessary for a sequential set of reactions a mixture of microbial cultures can be used. Thus, Gluconobacter oxydans containing glucose oxidase has been used in conjunction with saccharonyces cerevisiae cells containing periplasmic invertase or permeabilized Kluyveromyces marxianus cells containing intracellular β-galactosidase, in the fabrication of a sucrose and a lactose biosensor, respectively [Svitel (1998) Biotechnol. Appl. Biochem. 27:153-158]. Note, the major drawback of such an approach is die need to maintain at least two cultures of microorganisms on a single sensor which may prove problematic such as due to different nutritional needs.

Microbial biosensors based on light emission from luminescent bacteria are also utilized in analyte detection. Bioluminescent bacteria are found in nature, their habitat ranging from marine to terrestrial environments. Bioluminescent whole cell biosensors have also been developed using genetically engineered microorganisms for the monitoring of organic, pesticide and heavy metal contamination. The microorganisms used in these biosensors are typically produced with an exogenous plasmid into which a reporter gene under the control of an inducible promoter of interest is placed.

The use of non-specific stress responses as general indicators of deleterious conditions, is possible by employing a large variety of stress triggered genes (e.g., the heat shock response. Accordingly, E. coli strains containing the heat shock promoter grpE, dnaK or lon fused to the lux reporter increase bioluminescence in response to many chemicals [Van Dyk (1994) Appl. Environ. Microbiol. 60:1414-1420; Ben Israel (1998) Appl. Environ. Microbiol. 64:4346-4352].

However, all currently available whole-cell based biosensors are limited in detection to a closed list of materials and thus are incapable of broad applications.

While reducing the present invention to practice, the present inventor created a panel of genetically manipulated cells which can be used as biosensors in identification of a broad range of analytes thus overcoming the limitations inherent to prior art biosensors.

### SUMMARY OF THE INVENTION

According to one aspect of the present invention there is provided a population of cells comprising at least two subpopulations of cells, wherein a first subpopulation of the at least two subpopulation of cells includes a first reporter expression construct being expressible in a cell of the first subpopulation when exposed to a first analyte and whereas a second subpopulation of the at least two subpopulation of cells includes a second reporter expression construct being expressible in a cell of the second subpopulation when exposed to a second analyte.

According to another aspect of the present invention there is provided a device for detecting presence, absence or level of a substance in a sample, the device comprising a substrate being configured for supporting a population of cells including at least two subpopulations of cells, wherein a first subpopulation of the at least two subpopulations of cells includes a first reporter expression construct being expressible in a cell of the first subpopulation when exposed to a first analyte and whereas a second subpopulation of the at least two subpopulation of cells includes a second reporter expression construct being expressible in a cell of the second subpopulation when exposed to a second analyte, wherein each of the at least two subpopulations of cells is attached to the substrate in an addressable manner.

According to yet another aspect of the present invention there is provided a system for detecting presence, absence or level of a substance in a sample, the system comprising: (a) a device including a substrate being configured for supporting a population of cells including at least two subpopulations of cells, wherein a first subpopulation of the at least two subpopulations of cells includes a first reporter expression construct being expressible in a cell of the first subpopulation when exposed to a first analyte and whereas a second subpopulation of the at least two subpopulation of cells includes a second reporter expression construct being expressible in a cell of the second subpopulation when exposed to a second analyte, wherein each of the at least two subpopulations of cells is attached to the substrate in an addressable manner; (b) a detector for detecting expression from each of the first and second reporter expression constructs in the population of cells; and (c) a processing unit for obtaining and processing data representing the expression detected by the detector to thereby provide information relating to the presence, absence or level of the substance in the sample.

According to still further features in the described preferred embodiments the substrate, is configured as a multiwell matrix, whereas each well includes a culture of one of the at least two subpopulations of cells.

According to still further features in the described preferred embodiments a material of the substrate is selected from the group consisting of a glass, a polymer, a ceramic, a metal and a composite thereof.

According to still another aspect of the present invention there is provided a method of detecting presence, absence or level of a substance in a sample, the method comprising: (a) exposing a population of cells to the sample, the population of cells including at least two subpopulations of cells, wherein a first subpopulation of the at least two subpopulations of cells includes a first reporter expression construct being expressible in a cell of the first subpopulation when exposed to a first analyte and whereas a second subpopulation of the at least two subpopulation of cells includes a second reporter expression construct being expressible in a cell of the second subpopulation when exposed to a second analyte with the sample; and (b) analyzing expression of the reporter expression constructs in each of the at least two subpopulations of cells, to thereby detect presence, absence or level of the substance in the sample.

According to further features in preferred embodiments of the invention described below, the population of cells is eukaryotic cells.

According to still further features in the described preferred embodiments the population of cells is prokaryotic cells.

According to still further features in the described preferred embodiments each of the reporter expression construct includes a cis-acting regulatory element being operably fused to a reporter gene.

According to still further features in the described preferred embodiments the reporter genesis selected from a group consisting of a fluorescent protein, an enzyme and an affinity tag.

According to still further features in the described preferred embodiments the cis-acting regulatory element is a promoter.

According to still further features in the described preferred embodiments the promoter is selected from the group consisting of MipA, LacZ, GrpE, Fiu, MalPQ, oraA, nhoA, recA, otsAB and yciD.

According to still further features in the described preferred embodiments the cis-acting regulatory element is stress regulated.

According to still further features in the described preferred embodiments each of the first or the second analyte is independently selected from the group consisting of a condition and a substance.

According to still further features in the described preferred embodiments the condition is selected from the group consisting of a temperature condition and a radiation condition.

According to still further features in the described preferred embodiments the substance is a naturally occurring product or a synthetic product.

According to still further features in the described preferred embodiments each of the at least two subpopulations of cells is tagged.

According to still further features in the described preferred embodiments analyzing expression is effected by a pattern recognition software.

According to still further features in the described preferred embodiments the pattern recognition software is combined with neural network.

The present invention successfully addresses the shortcomings of the presently known configurations by providing novel populations of cells and, devices and systems which utilize such cell populations for analyte detection. Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described below. In case of conflict, the patent specification, including definitions, will control. In addition, the materials, methods, and examples are illustrative only and not intended to be limiting.

### BRIEF DESCRIPTION OF THE DRAWINGS

The invention is herein described, by way of example only, with reference to the accompanying drawings. With specific reference now to the drawings in detail, it is stressed that the particulars shown are by way of example and for purposes of illustrative discussion of the preferred embodiments of the present invention only, and are presented in the cause of providing what is believed to be the most useful and readily understood description of the principles and conceptual aspects of the invention. In this regard, no attempt is made to show structural details of the invention in more detail than is necessary for a fundamental understanding of the invention, the description taken with the drawings making apparent to those skilled in the art how the several forms of the invention may be embodied in practice.

In the drawings:

FIGS. 1a-c; are a schematic illustration depicting a general configuration of the biosensor of the present invention.

FIG. 2 is a scheme of the pDEW201 plasmid containing the luxCDABE operon from the terrestrial bacterium Photorhabdus luminescens. This plasmid was used for the luxCDABE library construction [VanDyk, T. K. (2001) LuxArray, a High-Density, Genomwide Transcription Analysis of Escherichia coli Using Bioluminescent Reporter Strains. Journal of Bacteriology 183:5496-5505].

FIG. 3a is an electronic presentation depicting a multi-repetition assay format.

FIGS. 3b-d are graphs depicting the parameters which were used to charcterize the panel's responses to selected toxicants in terms of intensity, sensitivity and kinetics. ΔLₘₐₓ is the maximal difference between the RLU produced by the induced and uninduced bacteria (FIG. 3b). RRₘₐₓ is the maximal ration obtained from dividing the induced RLU value with the control RLU (FIG. 3c). tₘₐₓ is the time points ΔLₘₐₓ and RRₘₐₓ values were produced. Cmax is the toxicant concentration which produced these responses (FIG. 3d). t₂₀₀ is the time when a response ratio of 2 is produced (FIG. 3c). EC200 is the toxicant concentration which induces the t₂₀₀ ratio.

FIGS. 4a-b are graphs depicting the response of oraA::luxCDABE to nitrogen mustard. RLU intensity (FIG. 4a) and response ratio (FIG. 4b) were examined in order to select the most suitable promoters for the panel.

FIG. 5 is an electronic presentation of the data loaded to the nn system in the NetMaker format. The input values were based on the RLU values measured in the panel after 30, 60, and 120 minutes as response to the Cₘₐₓ of the different toxicants. Output was chosen arbitrarily to be “DDVP”.

FIGS. 6a-c are graphs depicting the RLU values which were used to run the three nn systems. The nn was introduced with average RLU values collected from 40 repetitions of the panel members in response to five different toxicants and sterile LB 30, 60, and 120 minutes following exposure (FIG. 6a, FIG. 6b and FIG. 6c, respectively).

FIG. 7 is an electronic presentation of the resulting DP values as detailed in the BrainMaker software format. The left side of the screen shows the input data of the five promoters. The next column to the right shows the output values to each compound. These are the probabilities according to the trained nn system for the presence of each compound in the sample. In this case the nn detected the presence of DDVP in the sample based on the 120 minute nn system.

FIGS. 8a-c are graphs depicting the results of nn testing with RLU values collected from the panel in response to low toxicant concentrations. The graphs describe the nn DP values versus the toxicant concentration produced by the 30 (FIG. 8a), 60 (FIG. 8b) and 120 (FIG. 8c) minutes nns.

FIG. 9 is a graph depicting survival of E. coli MG1655 grown in LB or M-9 with or without 0.5 M NaCl immediately after freeze drying.

FIG. 10 is a graph depicting survival of E. coli MG1655 grown in LB with or without 0.5 M NaCl immediately after freeze-drying or after storing at different temperature conditions.

FIG. 11 is a graph depicting survival of E. coli MG1655 grown in LB or M-9 with or without 0.5 M NaCl immediately after high temperature drying.

FIG. 12 is a graph depicting survival and stability of E. coli MG1655 grown in M-9 with or without 0.5 M NaCl immediately after high temperature drying or after storing at different temperature conditions.

FIGS. 13a-b are graphs depicting expression of grpE::Lux (FIG. 13a) and recA::Lux (FIG. 13b) as a function of the concentration of fresh cells.

FIGS. 14a-b are graphs depicting expression of grpE:.Lux (FIG. 14a) and recA::Lux (FIG. 14b as a function of the concentration of dry cells.

FIG. 15 is a graph depicting luciferase activity in grpE::Lux and recA:.Lux rehydrated or fresh cells.

FIG. 16 is a graph depicting induction of recA::Lux expression in dry cells as a function of sample volume and fixed cell concentration (i.e., 5×10⁸ cells/ml).

FIGS. 17a-b are graphs depicting induction of recA::Lux expression in dry (FIG. 17a) or fresh (FIG. 17b) cells as a function of cell concentration and fixed sample volume (i.e., 0.5 μl).

FIG. 18 is a graph depicting induction of recA::Lux in dry cells expressing same as a function of cell concentration (higher range than used in FIG. 17a) and a fixed sample volume (i.e., 0.5 μl).

FIGS. 19a-b are graphs depicting the growth of recA:.Lux transformed dry cells in LB or M9 medium following storage at −20° C. (FIG. 19a) or −4° C. (FIG. 19b).

FIGS. 20a-c are simplified illustrations of a system for detecting the analyte using optical signal, according to a preferred embodiment of the present invention;

FIG. 21 is a simplified illustration of a light detector, according to a preferred embodiment of the present invention;

### DESCRIPTION OF THE PREFERRED EMBODIMENTS

The present invention is of populations of cells, which can be utilized in devices and systems configured for analyte detection.

The principles and operation of the present invention may be better understood with reference to the drawings and accompanying descriptions.

Before explaining at least one embodiment of the invention in detail, it is to be understood that the invention is not limited in its application to the details set forth in the following description or exemplified by the Examples. The invention is capable of other embodiments or of being practiced or carried out in various ways. Also, it is to be understood that the phraseology and terminology employed herein is for the purpose of description and should not be regarded as limiting.

Biosensors are fast becoming the preferred approach for analyte detection in cases where rapid qualification and/or quantification of substances present in a liquid or gaseous samples is desired.

Although numerous examples of biosensors exist in the art including enzyme-based biosensors, antibody-based biosensors and whole cell-based biosensors, such biosensors are limited by instability of the biological component, irreversibility, costs of production and limited ability to identify broad range of analytes.

To overcome such limitations, the present inventors have devised and constructed a reporter-expressing cell population which is composed of discrete subpopulations each capable of expressing the reporter in response to a different analyte or groups of analytes. When exposed to an analyte, the various subpopulations produce a specific expression pattern which forms a signature profile specific to the analyte present in the sample. As is further detailed in the Examples section which follows, to enable such analyte specific expression, the present inventors carefully selected a group of promoters which can be activated by different analytes from a number of promoter libraries. It is postulated herein that by utilizing a broad range of physiologically-responsive promoters, one increases an ability of a cell population transformed with reporter constructs containing such promoters to uniquely respond (via unique reporter expression patterns) to each of a broad range of analytes.

The present biosensing population of cells and a biosensing device using same stands in sharp contrast to U.S. Pat. No. 6,377,721 which teaches analyte detection using a biosensor which employs a limited number of cell populations (e.g., wild-type or genetically manipulated) which are capable of responding to very specific analytes (e.g., metals).

Thus, according to one aspect of the present invention there is provided a population of cells which can be utilized in analyte detection. The population of cells is composed of at least two subpopulations of cells; the first subpopulation of cells includes a first reporter expression construct which is capable of reporter expression when the cells of this subpopulation are exposed to a first analyte. The second subpopulation of cells includes a second reporter expression construct which is capable of reporter expression when the cells of the second subpopulation are exposed to a second analyte. Although the use of two distinct subpopulation constructed with accordance to the teachings of the present invention represents a technological advance in the field of biosensors, it will be appreciated that the use of three, four, five, six or even more subpopulations is preferred since it enables detection of an even broader range of analytes.

As used herein “population of cells” refers to prokaryotic or eukaryotic cells which can be genetically modified (in a transient or stable manner) to express exogenous polynucleotides.

Examples of prokaryotic cells which can be used in accordance with this aspect of the present invention include but are not limited to bacterial cells, such as Pseudomonas, Bacillus, Bacteriodes, Vibrio, Yersinia, Clostridium, Mycobacterium, Mycoplasma, Coryynrebacterium, Escherichia, Salmonella, Shigella, Rhodococcus, Methanococcus, Micrococcus, Arthrobacter, Listeria, Klebsiella, Aeromonas, Streptomyces and Xanthomonas.

Examples of eukaryotic cells which can be used in accordance with this aspect of the present invention include but are not limited to cell-lines, primary cultures or permanent cell cultures of fungal cells such as Aspergillus niger and Ustilago maydis [Regenfelder, E. et al. (1997) EMBO J. 16:1934-1942], yeast cells (see U.S. Pat. Nos. 5,691,188, 5,482,835 and Example 5 of the Examples section which follows), such as Saccharomyces, Pichia, Zygosaccharomyces, Trichoderma, Candida, and Hansenula, plant cells, insect cells, nematoda cells such as c. elegans, invertebrate cells, vetebrate cells and mammalian cells such as fibroblasts, epithelial cells, endothelial cells, lymphoid cells, neuronal cells and the like. Cells are commercially available from the American Type Culture Co. (Rockville, Md.).

As mentioned hereinabove the population of cells of this aspect of the present invention includes at least two subpopulations of cells. However, it is appreciated that the more subpopulations included in the cell population of the present invention the higher the chances of such a cell population to accurately identify analytes present in a sample exposed thereto. Construction of a cell population in accordance with the present invention is described in details in Example 1 of the Examples section.

As mentioned hereinabove, each subpopulation of cells includes a reporter expression construct, which expresses a detectable reporter molecule when the cell is exposed to an analyte.

As used herein “reporter expression construct” refers to a vector which includes a polynucleotide sequence encoding a reporter. The reporter expression construct of this aspect of the present invention can be designed to randomly integrate into the genome of the cell, such that expression of the reporter polypeptide is governed by an endogenous regulatory element which is inducible by an analyte.

According to a preferred embodiment of this aspect of the present invention, the polynucleotide sequence is positioned in the construct under the transcriptional control of at least one cis-regulatory element suitable for directing transcription in the subpopulation of cells upon exposure to an analyte.

As used herein a “cis acting regulatory element” refers to a naturally occurring or artificial polynucleotide sequence, which binds a trans acting regulator and regulates the transcription of a coding sequence located down-stream thereto. For example, a transcriptional regulatory element can be at least a part of a promoter sequence which is, activated by a specific transcriptional regulator or it can be an enhancer which can be adjacent or distant to a promoter sequence and which functions in up regulating the transcription therefrom.

It will be appreciated that the cis-acting regulatory element of this aspect of the present invention may be stress regulated (e.g., stress-regulated promoter), which is essentially activated in response to cellular stress produced by exposure of the cell to, for example, chemicals, environmental pollutants, heavy metals, changes in temperature, changes in pH, as well as agents producing oxidative damage, DNA damage, anaerobiosis, and changes in nitrate availability or pathogenesis.

Examples of promoters which are preferably used in accordance with this aspect of the present invention include, but are not limited to, MipA, LacZ, GrpE, Fiu, MalPQ, oraA, nhoA, recA, otsAB and yciD.

A cis acting regulatory element can also be a translational regulatory sequence element in which case such a sequence can bind a translational regulator, which up regulates translation.

The term “expression” refers to the biosynthesis of a gene product. For example, in the case of the reporter polypeptide, expression involves the transcription of the reporter gene into messenger RNA (mRNA) and the translation of the mRNA into one or more polypeptides.

As used herein “reporter polypeptide” refers to a polypeptide gene product, which, can be quantitated either directly or indirectly. For example, a reporter polypeptide can be an enzyme which when in the presence of a suitable substrate generates chromogenic products. Such enzymes include but are not limited to alkaline phosphatase, β-galactosidase, β-D-glucoronidase (GUS), luciferase and the like. A reporter polypeptide can also be a fluorescer such as the polypeptides belonging to the green fluorescent protein family including the green fluorescent protein, the yellow fluorescent protein, the cyan fluorescent protein and the red fluorescent protein as well as their enhanced derivatives. In such a case, the reporter polypeptide can be quantified via its fluorescence, which is generated upon the application of a suitable excitatory light. Alternatively, a polypeptide label can be an epitope tag, a fairly unique polypeptide sequence to which a specific antibody can bind without substantially cross reacting with other cellular epitopes. Such epitope tags include a Myc tag, a Flag tag, a His tag, a Leucine tag, an IgG tag, a streptavidin tag and the like. Further detail of reporter polypeptides can be found in Misawa et al. (2000) PNAS 97:3062-3066.

As used herein the term “analyte” refers to a molecule or a mixture of molecules in a liquid, gaseous or aerosol medium. It will be appreciated that molecules can be completely soluble in a liquid medium, alternatively they may be in a colloidal state. Thus analytes in liquid medium may be in solution or carried by the liquid medium.

Examples of analytes include, but are not limited to, small molecules such as naturally occurring compounds (e.g., compounds derived from plant extracts, microbial broths, and the like) or synthetic compounds having molecular weights of less than about 10,000 daltons, preferably less than about 5,000 daltons, and most preferably less than about 1,500 daltons, electrolytes, metals, peptides, nucleotides, saccharides, fatty acids, steroids and the like. Analytes typically include at least one functional group necessary for biological interactions (e.g., amine group, carbonyl group, hydroxyl group, carboxyl group).

It will be appreciated that in certain aspects of the present invention the reporter expression construct may be expressed in response to a growth condition. Examples of such conditions include, but are not limited to temperature, humidity, atmospheric pressure, contact surfaces, radiation exposure (such as, γ-radiation, UV radiation, X-radiation).

As mentioned hereinabove, each reporter expression construct is expressed in a subpopulation of cells upon exposure to a distinct analyte or groups of analytes. It will be appreciated however, that since several unrelated analytes can lead to the same effect on a cell, an expression construct of the present invention can also be expressed albeit at lower efficiency upon exposure to other analytes. Such partial overlap between the different reporter expression constructs is desirable since it will increase the detection range of the population of the present invention to thereby enable identification of numerous analytes even at low concentration levels. For example, if a first analyte induces reporter expression from one subpopulation it may be difficult to distinguish it from a second unrelated analyte which also induces expression in the same subpopulation. However, if several cell subpopulations are induced by a first analyte (each subpopulation expressing a unique level of the reporter) the likelihood that the same subpopulations will also react with the same expression pattern upon exposure to a second analyte is remote.

Dependent on the host cell used, the reporter expression construct can include additional elements. For example, polyadenylation sequences can also be added to the reporter expression construct in order to increase the translation efficiency of a reporter polypeptide expressed from the expression construct of the present invention. Two distinct sequence elements are required for accurate and efficient polyadenylation: GU or U rich sequences located downstream from the polyadenylation site and a highly conserved sequence of six nucleotides, AAUAAA, located 11-30 nucleotides upstream. Termination and polyadenylation signals that are suitable for the present invention include those derived from SV40.

In addition to the elements already described, the expression construct of the present invention may typically contain other specialized elements intended to increase the level of expression of cloned nucleic acids or to facilitate the identification of cells that carry the recombinant DNA. For example, a number of animal viruses contain DNA sequences that promote the extra chromosomal replication of the viral genome in permissive cell types. Plasmids bearing these viral replicons are replicated episomally as long as the appropriate factors are provided by genes either carried on the plasmid or with the genome of the host cell.

The construct may or may not include a eukaryotic replicon. If a eukaryotic replicon is present then the vector is amplifiable in eukaryotic cells using the appropriate selectable marker. If the construct does not comprise a eukaryotic replicon, no episomal amplification is possible. Instead, the recombinant DNA integrates into the genome of the engineered cell, where the promoter directs expression of the desired nucleic acid.

The reporter expression construct can be introduced into the cell using a variety of molecular and biochemical methods known in the art. Examples include, but are not limited to, transfection, conjugation, electroporation, calcium phosphate-precipitation, direct, microinjection, liposome fusion, viral infection and the like. Selection of a suitable introduction method is dependent upon the host cell and the type of construct used.

Since the response of each subpopulation of the cell population of the present invention to an analyte needs to be assesed independently in order to generate a signature expression pattern, each cell of each subpopulation is preferably tagged with a distinct tag unique to the subpopulation. The tag may be for example, a fluorophoric or chromophoric dye compound which may be detected using a microscope. Such dyes are commercially available such as from Molecular Probes (Eugene, Oreg., USA). Alternatively, cells can be naturally fluorescing or genetically engineered to fluoresce. Molecular tags can also be used. Such tags may be detected by amplification methods, such as PCR.

The above described cell population can be employed in a variety of applications. For example, in the environmental field the cell population of the present invention can be employed to detect the presence of pollutants such as halogenated hydrocarbons (used as pesticides), polycyclic aromatic hydrocarbons (carcinogenic compounds), acrylamide, acrylic acid and acrylonitrile, organophosphorous compounds (used as pesticides, insecticides, and chemical warfare agents), nitroaromatic compounds, such as nitrophenols, picric acid, trinitrotoluene (used as xenobiotics present in wastes of chemical armament plants as in civil factories for dye, pesticide, and other chemical manufacturing). Alternatively, the cell population of the present invention can be employed in the food and fermentation industries, where there is a need for quick and specific analytical tools. Analysis is needed for monitoring nutritional parameters, food additives, food contaminants, microbial counts, shelf life assessment, compliance with specifications or regulations, and other olfactory properties like smell and odor. In pharmaceuticals and medicine the cell population of the present invention can be used for drug identification and qualification (e.g., determination of active ingredients in pharmaceutical formulations]. The cell populations of the present invention can also be used for detecting narcotics and explosives such as trinitrotoluene (TNT), cyclonite (RDX), pentaerythritol tetranitrate (PETN) C-4 class explosives, and combinations thereof [Yinon, Y. and Zitrin, S. (1993) Modern Methods and Applications in Analysis of Explosives, John Wiley & Sons, Ltd., Sussex, U. K.].

Thus, according to another aspect of the present invention there is provided a method of detecting presence, absence or level of an analyte in a sample.

As used herein the term “sample” refers to any liquid, gas or aerosol specimen.

The method of this aspect of the present invention is effected by exposing the above-described population of cells to the sample and analyzing expression of the reporter expression constructs in each of the at least two subpopulations of cells, to thereby detect presence, absence or level of the substance in the sample.

To simplify analysis, each subpopulation of cells is aliquoted to a different culture dish or to a different well of multiwell plates at a pre-calibrated density. A number of controls may be included in the assays. For example, a subpopulation of cells which is designed to constitutively express the reporter polypeptide is preferably for qualifying the reagents used (e.g., substrate hydrolizability). Alternatively, or additionally, naive subpopulation of cells or cells tranfected with an empty reporter plasmid, not including the reporter polynucleotide encoding the reporter polypeptide, may be included for monitoring background signal.

The sample can be either contacted with or introduced into the cell, using molecular or biochemical methodologies well known in the art. Examples include but are not limited to, transfection, conjugation, electroporation, calcium phosphate-precipitation, direct microinjection, liposome fusion and the like.

Following an appropriate time of incubation in the presence of the sample reporter, activity from each reporter expression construct is analyzed. Thus, for example, when the reporter activity can be visualized, a magnifying optical device, typically equipped with filters for detection of the reporter molecule, is used for analyzing expression of the reporter expression constructs in each subpopulations of cells subjected to sample treatment.

Basically, reporter activity occurrence indicates presence of an analyte, signal intensity provides a measure for analytes level in the sample and the pattern of signal reflects the identity of the analyte in the sample.

Analysis of measurement data is preferably effected using imaging software to process images of the cell population using clustering or pattern recognition methods which can be based, for example, on neural network techniques. Examples of clustering and pattern recognition methods which are contemplated in the present embodiment include, but are not limited to, nearest neighbor algorithm, back-propagation algorithm, K-means algorithm, density estimation algorithm and methods based on graph theory. The analysis of measurement data may also be effected using other statistical methods, such as, but not limited to, factor analysis, principle component analysis, auto-correlation method, burst analysis and the like. A representative example of toxicant recognition using neuronal networks is given in Example 2 of the Examples section that follows.

Preferably the method of the present invention is used for high throughput analyte detection using a plurality of subpopulations of cells (e.g., 6 subpopulations). To facilitate use the cell subpopulations of the present invention are preferably used as the sensing element in a biosensor device.

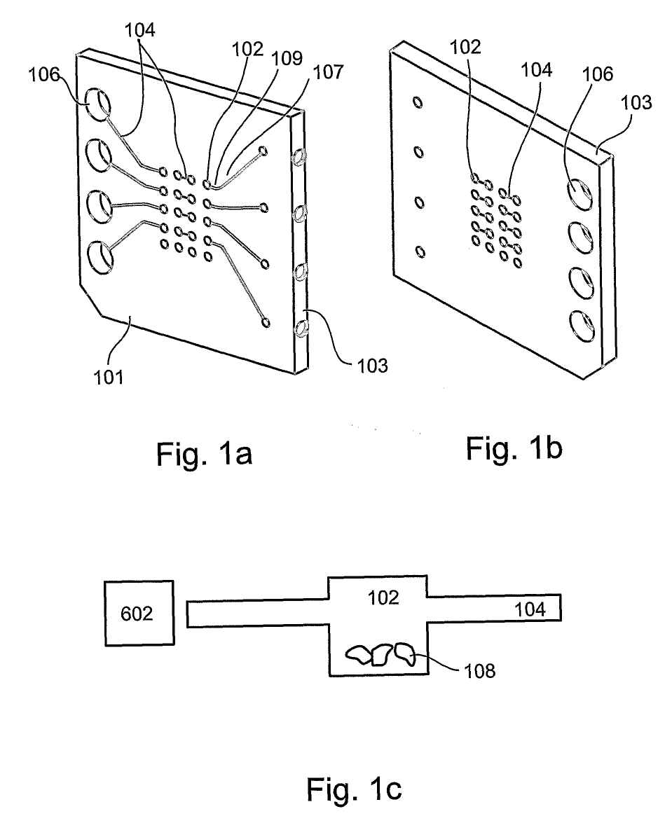

Referring now to the drawings, FIGS. 1a-c illustrate a device for detecting presence, absence or level of an analyte in a sample which is referred to herein as device 10.

Device 10 includes a substrate 101 configured for supporting population of cells 108 (which includes the various subpopulations described hereinabove), in an addressable manner, i.e., enabling identification of each discrete subpopulation of cells. Substrate 101 is preferably fabricated from a material, which can accommodate discrete individual sites (e.g., wells, chambers, etc.) configured for containment, attachment or association of population of cells 108. Examples of material suitable for fabrication of substrate 101 include, but are not limited to, glass (including modified or functionalized glass), plastics (e.g., acrylics, polystyrene, polypropylene, polyethylene, polybutylene, polyurethanes), polysaccharides, nylon, nitrocellulose, resins, silica, silica-based materials (e.g., silicon), carbon, metals, inorganic glasses, optical fiber bundles (see U.S. Pat. No. 6,377,721) and the like. Substrate 101 is preferably selected such that it allows optical detection of a signal generated by the various cell subpopulations contained therein or attached thereto. Substrate 101 is typically planar, although other configurations can also be used in device 10. For example, three dimensional configurations of substrate 101, can be generated by embedding the cells of the various subpopulations in a porous block of plastic that allows detection of a signal generated by these cells. Similarly, population of cells 108 can be placed on the inside surface of a tube, for flow through sample analysis to minimize sample volume.

At least one surface 103 of substrate 101 is fabricated with, or is modified (e.g., etched) to include discrete locations (e.g., chambers, wells) 102 which are configured or modified so as to enable holding one or more subpopulations of population of cells 408.

Locations 102 may be regularly or randomly distributed in or on surface 103. A preferred embodiment utilizes a regular pattern of locations such that the sites may be addressed using an X-Y coordinate system.

In a preferred embodiment, locations 102 are formed as microwells 107, i.e. depressions in the surface of the substrate. This may be done as is generally known in the art using a variety of techniques, including, but not limited to, photolithography, stamping techniques, pressing, casting, molding, microetching, electrolytic deposition, chemical or physical vapor deposition employing masks or templates, electrochemical machining, laser machining or ablation, electron beam machining or ablation, and conventional machining. As will be appreciated by those skilled in the art the technique used will depend on the composition and shape of the substrate, as well as on sample volume. Preferred sample volume is between about 1 fl-10 μl, more preferably between 1 fl-1 μl, even more preferably between about 1 fl -0.5 μl, even more preferably between about 1 fl-100 nl, even more preferably between about 1 fl-10 nl, even more preferably between about 1 fl-1 nl.

In addition to, or as an alternative to the microwells described above, locations 102 can also include biological or chemical groups or compounds, that can be used to attach, (either covalently or non-covalently) the cells of the invention at locations 102. Examples of chemical groups include amino groups, carboxy groups, oxo groups and thiol groups.

For example, addition of charged groups on surface 103 can be used for the electrostatic attachment of population of cells 108. Alternatively the addition of chemical functional groups that renders the locations 102 differentially hydrophobic or hydrophilic will result in association of population of cells 108 at locations 102 on the basis of hydroaffinity. Any of these groups or compounds can be applied in a desired pattern to surface 103 so as to enable formation of locations 102.

Alternatively, binding ligands or binding partner pairs, including, but not limited to, antigen/antibody pairs, enzyme/substrate or inhibitor pairs, receptor-ligand pairs, carbohydrates and their binding partners (ectins, etc.). may be used to enable formation of locations 102 in a desirable pattern.

Device 10 also includes one or more sample ports 106, each being in fluid communication with locations 102 via channels 104. Sample ports 106 serve for feeding sample 105 (gas or liquid) through channels 104 and into locations 102. When sample 105 is in a gaseous state, its components (e.g., organic components) are preferably bound to an aqueous phase prior to the feeding of sample port 106.

Surface 103 may also be coated with a material 109 which supports cell growth. Materials known to support cell growth or adhesion include biological compatible materials, including, but not limited to, fibronectin, any number of known polymers including collagen, polylysine and other polyamino acids, polyethylene glycol and polystyrene, growth factors, hormones, cytokines, etc. Similarly, binding ligands as outlined above may be coated onto the surface of the wells. In addition, coatings or films of metals such as gold, platinum or palladium may also be employed in order to facilitate chemical modification of the surface (e.g., thiolization) as described above.

The size of microwell 107, which is the presently preferred configuration of locations 102, is selected according to the type and number of cells used (i.e., eukaryotic fibroblast cells are about 20 μm in size, neurons may reach 1080 μm in size, E. coli are about 1-2 μm in size

Population of cells 108 can be applied to surface 103 using any method of adsorption, entrapment, covalent binding, cross-linking or a combination thereof known in the art, although it will be appreciated that the specific method(s) utilized will depend on the nature and type of locations 102. Since the present invention relies on the activity of viable cells, gentle immobilization techniques such as entrapment and adsorption are preferably utilized. Typically the cells are retained in close proximity to the detector by using membranes such as a dialysis membrane. In general the outer membrane is chemically and mechanically stable, with a thickness of 10-15 μm and a pore size of 0.1-1 μm. Preferably used are pore trace membranes made of polycarbonate or polyphthalate. Other immobilization methods are described in U.S. Pat. No. 6,692,696.

Cells may be immobilized to provide an array of at least 2 cells, although arrays having high cell density are preferred since signals generated from such cells increase in proportion to the number of cells utilized. Thus, cells may be applied/cultured on surface 103, at very low density (e.g., 10 to 1000 cells/cm²), low density (e.g., 1,000 to 5,000 cells/cm²), moderate density (e.g., 10,000 to 100,000 cells/cm²), high density (e.g., 100,000 to 10,000,000 cells/cm²) or very high density (e.g., 10,000,000 to 2,000,000,000 cells/cm²),

Cells are maintained viable on substrate 101 using any growth medium which matches the nutritional needs of the cells used [see ATCC quality control methods for cell lines (2^{nd} ed.) American Type Culture Co. (Rockville, Md.)]. Increasing intracellular compatible solute concentration [e.g., by active import from the intracellular environment (e.g., uploading with non-metabolizable sugars) or by inducing autosynthesis (e.g., genetic engineering, growth in high salinity medium)] may be preferred since it is well established that accumulation of compatible solutes, may provide enhanced resistance to freezing and drying. Examples of compatible solutes include, but are not limited to, glycine, proline, hydroxyectoine and trehalose. Measures are preferably taken to use subpopulations of cells which exhibit substantially similar growth rates, so as to obtain an equilibrated signal from the different reporter constructs upon exposure to analytes.

Once immobilized the viability of the cells is preferably tested using cell biology, molecular and/or biochemical methods which are well known in the art, or using commercially available viability kits (Molecular Probes, Eugene, Oreg., USA). Additionally or alternatively, viability is tested by analyzing transcriptional response to known analytes.

When sample 105 is in a fluid state, locations 102 are preferably configured as reaction chambers 109. Reaction chambers 109 are preferably addressable so as to allow addressable monitoring thereof, as further detailed hereinunder.

Given that a sufficient number of cells are utilized the intensity of the signal, generated by population of cells 108, typically depends on the amount of detectable analyte being in contact therewith. For a given sample fluid having a given concentration the intensity of the signal is proportional to the amount of sample fluid present in reaction chambers 109. To optimize correlation between the intensity of the signal and the concentration of the analyte in sample fluid 105, reaction chambers 102 are preferably of substantially equal volume.

Fluid channels 104 are preferably microfluidic channels. Transport of sample 105 from sample port 106 through channels 104 and into or onto reaction chambers 109 can be effected using a variety of methods which are known in the art. Preferably sample transport is effected in a manner which enables provision of an equal fluid volume to each of reaction chambers 109.

There are many techniques for actuating fluid transport through microchannels. One example of a mechanism suitable for transporting sample 105 to reaction chambers 109 is illustrated in FIG. 1C (indicated by 602). Mechanism 602 can be a pump or an injector capable of pumping or injecting a sample fluid through channels 104 and into or onto reaction chambers 109. Mechanism 602 can be placed on or in device 10 or not, depending on considerations such as costs, size of substrate 101 and the like. In any case, mechanism 602 is in fluid communication with sample port 106, reaction chambers 109 or microwells 107 to enable sample 105 delivery to microwells 107.

Several Examples of micropumps or microinjectors which can be utilized in mechanism 602 are known in the art (e.g., micro-pump). In any event, mechanism 602 preferably enables sample 105 delivery by applying a negative pressure to channels 104 reaction chambers 109 thereby delivering sample 105 from sample port 106 to reaction chambers 109.

As used herein “negative pressure” refers to a pressure value, which is smaller than a pressure value in a reference volume. For example, with respect to sample port 106, “negative pressure” refers to a pressure value which is smaller than the pressure value in sample port 106. The terms “negative pressure” and “under-pressure” are interchangeably used herein.

Mechanism 602 can include one or preferably several micro-pumps, which are capable of generating negative pressure in locations 102 with respect to the pressure in sample port 106 when actuated.

As is mentioned hereinabove, cell population 108 of the present invention responds to presence of a particular analyte with expression of a detectable reporter protein (preferably producing a calorimetric or fluorogenic signal). Thus, to enable detection of such cell generated signals, device 10 of the present invention forms a part of a system capable of detecting presence, absence or level of an analyte by qualifying and optionally quantifying optical signals generated by each subpopulation of cell population 108 of device 10.

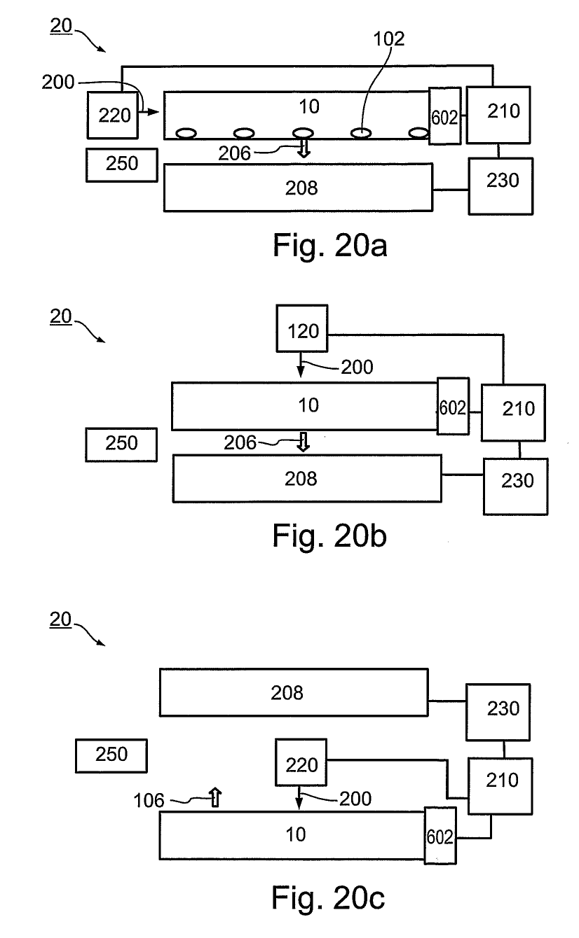

Reference is now made to FIGS. 20a-c, which provide simplified illustrations of system 20. In its simplest configuration system 20 includes a detecting device, e.g., device 10 and a light detector 208 for detecting an optical signal 206 generated by population of cells 108 (not shown in FIGS. 20a-c). System 20 may further include mechanism 602. According to a preferred embodiment of the present invention system 20 further includes a control unit 210 and a data processor 230. Control unit 210 sends control signals to components of system 20 for timing their operation. For example, control unit 210 may send activating and deactivating signals to light source 220 or mechanism 602. Data processor 230 serves for processing signals received from detector 208 and thus is in data communication therewith.

System 20 may further comprise a power source 202 for supplying energy thereto. Power source 202 can be fixed or portable, replaceable or rechargeable, integrated, with or being an accessory to system 20. Examples of fixed power sources include, but are not limited too, a power source from a wall socket and a fixed voltage generator. Examples of a mobile power sources include, but are not limited too, an electrochemical cell (e.g., a battery) and a mobile a voltage generator.

When power source 202 is portable, it can be implemented in device 10, data processor 230, light detector 108 or any other component of system 20. In this embodiment, power source 202 can be, for example, a traditional secondary (rechargeable) battery, a double layer capacitor, an electrostatic capacitor, an electrochemical capacitor, a thin-film battery (e.g., a lithium cell), a microscopic battery and the like. The type and size of power source 202 as well as the amount of energy stored therein may vary, depending on the required power and, in some embodiments, on the component in which power source 202 is implemented. For example, when data processor 230 is a portable computer, power source 202 can be an internal battery of the portable computer.

Detector 208 receives optical signal 206 from cells 108 and converts signal 206 into electronic signals (analog or preferably digital) which in turn can be received and analayzed, for example, by data processor 230. Detector 208 preferably detects optical signals 206 simultaneously from several locations 102. More preferably, detector 206 detects optical signals 206 simultaneously from all locations 102.

Data processor 230 is preferably designed to include software for determining the presence, absence or concentration of the analyte in the sample. For example, data processor 230 can determine whether or not the sample fluid is toxic and send an appropriate sensible signal to the user which can monitor the sensible signal, e.g., using a display. Data processor 230 can also calculate the concentration of the analyte in the sample fluid and provide the user with the information desired.

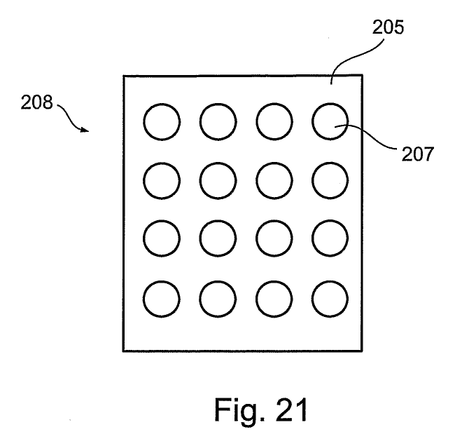

Reference is now made to FIG. 21, which is a simplified illustration of detector 208, according to a preferred embodiment of the present invention. Detector 208 preferably comprises a matrix 205 having a plurality of addressable elementary units 207, each being capable of converting light into an electrical signal. Each elementary unit is allocated to a specific location 102. When optical signal 206 originating from a particular location 102 impinges on matrix 205 the respective elementary unit generates a signal, which can then be analyzed by data processor 230. The signal generated by elementary units 207 preferably includes imagery information so as to allow attributing each signal to a respective location 102 thereby providing an image thereof. Thus, according to a preferred embodiment of the present invention detector 208 is capable of providing an image of signals 206 generated from the various cell subpopulations of cell population 108.

Several types of elementary detection units are contemplated herein. For example, elementary units 207 can be positive-intrinsic-negative (PIN) photodiodes. A PIN photodiode is a device having a large, neutrally doped intrinsic region sandwiched between p-doped and n-doped semiconducting regions. A PIN diode exhibits an increase in electrical conductivity as a function of the intensity, wavelength and modulation rate of incident radiation. The avalanche photodiode, is preferably used in accordance with the present invention since it is capable of generating an amplified current by avalanche multiplication in which electrons, initially generated by the incident light, accelerate and collide with other electrons.

Detector 208, which incorporates PIN photodiodes or avalanche photodiodes enables accurate monitoring of intensity as well as the wavelength of optical signal 206.

According to an alternative embodiment, detector 208 employs a charge-coupled device (CCD), in which elementary units 207 are silicon chips. When light hits the silicon chip, electrons are released from the crystalline structure of the silicon and deposited into small units or wells. Once the image is captured the electrons in the wells are sent into a recorder where they are counted.

Detector 208 can also utilize at least one photomultiplier for amplifying optical signal 206. Typically, a photomultiplier is a vacuum tube including a photocathode which is capable of converting light into electrons, by virtue of the photoelectric effect, an electron multiplier and an anode. When light enters the photocathode the photocathode electrons are emitted into the vacuum and then directed by a system of focusing electrode towards the electron multiplier. The electron multiplier is a string of successive electron absorbers with enhanced secondary emission hence multiply the numbers of electrons. The amplification of the electron multiplier can reach eight orders of magnitude. Once multiplied the electrons are collected by the anode as an output signal. Because of the high secondary-emission multiplication the photomultiplier provides extremely high sensitivity and low noise.

According to yet another alternative embodiment, detector 208 employs complementary metal oxide semiconductor (CMOS) technology. The advantage of using the CMOS technology is that the elementary units and various quantification parts can be integrated into a single device, which may be compact and simple to operate. Such CMOS are commercially available such as for example the ACS-1394 fire-wire camera based on the ACS-1024 CMOS Image Sensor manufactured by Photonics Vision Systems, or IBIS4 CMOS Image Sensor manufactured by Fill Factory http://www.fillfactory.com). Further description of a CMOS imaging sensor which can be used as detector 208, is provided in the Examples section which follows.

According to a preferred embodiment of the present invention, system 20 further includes at least one temperature control unit 150, for controlling the temperature of system 20. For example, temperature control unit 250, can monitor and adjust the temperature of device 10, detector 208 and/or light source 220 so as to optimize: their operation. This is of special significance since device 10 includes viable cells which require stable temperature for their growth and function of about 30-37° C. dependent on the type of cells used. Temperature control unit 250 can be, for example, a thermoelectric device.

A thermoelectric device is a device that either converts heat directly into electricity or transform electrical energy into pumped thermal power for heating or cooling. Such a device is based on thermoelectric effects involving relations between the flow of heat and electricity through solid bodies. Generally, a thermoelectric device comprises at least one pair of dissimilar metals. When the device is used for cooling or heating, a potential difference is applied on the dissimilar metals and heat is pumped from one metal to the other. When the device is used for converting heat to electricity (e.g., for the purpose of monitoring the temperature of an object relative to a reference environment) the two metals are kept at different temperatures, and a potential difference is produced across.

Other temperature control units include, but are not limited to, liquid coolers, gas coolers, blowers and the like.

To generate optical signal 206 system 20 also includes a light source 220 which is capable of generating light of a wavelength or wavelengths suitable for excitation of the reporter polypeptide produced by the various subpopulations of cell population 108. Light source 220 can be positioned in a number of points with respect to device 10, depending on the relative angle between the detected portion of optical signal 206 and excitation light 200. For example, in one embodiment, illustrated in FIG. 20a, light source 220 is positioned on the side of device 10, such that the detected portion of optical signal 206 is substantially perpendicular to excitation light 200. In another embodiment, illustrated in FIG. 20b, light source 220 is positioned above or below device 10 in a manner such that device 10 is positioned between light source 220 and light detector 208. In this embodiment the detected portion of optical signal 206 is substantially parallel to excitation light 200. In an alternative embodiment, illustrated in FIG. 20c, light source 220 is positioned between device 10 and light detector 208. In this embodiment the detected portion of optical signal 106 is substantially anti-parallel to excitation light 200.

It is to be understood that, although FIGS. 20a-b show light detector 208 positioned below device 10, this configuration is not to be considered as limiting.

For long-term storage of the cell-population of the present invention either as is, or when attached to a substrate (i.e., biosensor, e.g., device 10), drying of the cellular component is preferably effected. Drying formulations may include bulking agents, cryoprotectants, lyoprotectants, sugars to be preferably present in both the inside and outside of the cells. Two drying formulations are described in the Examples section which follows.

A number of drying methods are known in the art. Examples include, but are not limited to, freeze-drying, high temperature drying, air drying, spray drying, column drying. Typically, in freeze drying vacuum is applied following sample freezing The reduced pressure causes ice crystals to sublimate from the samples thereby promoting their drying. Samples are preferably kept frozen until sufficient low water content is achieved. In high temperature drying, vacuum is applied to samples that are maintained in higher than 0° C. (e.g., 20-30° C.), water then boils and removed quickly leaving the samples in a glassy foamed state.

In either case, upon termination of the primary drying step, which removes the bulk water, a secondary drying step is performed, essentially to achieve a sufficiently low water content to ensure low term stability and functionality. Typically, dry samples exhibit higher stability, though a too-low residual water content may be damaging. This secondary drying step involves an increase in sample temperature (e.g., 10-60° C.), which removes most remaining water. Selection of the drying protocol depends on the type of cells used and the assay performed. Such selection procedure is described in Example 4 of the Examples section which follows.

Additional objects, advantages, and novel features of the present invention will become apparent to one ordinarily skilled in the art upon examination of the following examples, which are not intended to be limiting. Additionally, each of the various embodiments and aspects of the present invention as delineated hereinabove and as claimed in the claims section below funds experimental support in the following examples.

### EXAMPLES

Reference is now made to the following examples, which together with the above descriptions, illustrate the invention in a non limiting fashion.

Generally the nomenclature used herein and the laboratory procedures utilized in the present invention include molecular, biochemical, microbiological and recombinant DNA techniques. Such techniques are thoroughly explained in the literature. See, for example, “Molecular Cloning: A laboratory Manual” Sambrook et al., (1989); “Current Protocols in Molecular Biology” Volumes I-III Ausubel, R. M., ed. (1994); Ausubel et al., “Current Protocols in Molecular Biology”, John Wiley and Sons, Baltimore, Md. (1989); Perbal, “A Practical Guide to Molecular Cloning”, John Wiley & Sons, New York (1988); Watson et al., “Recombinant DNA”, Scientific American Books, New York; Birren et al. (eds) “Genome Analysis: A Laboratory Manual Series”, Vols. 1-4, Cold Spring Harbor Laboratory Press, New York (1998); methodologies as set forth in U.S. Pat. Nos. 4,666,828; 4,683,202; 4,801,531; 5,192,659 and 5,272,057; “Cell Biology: A Laboratory Handbook”, Volumes I-III Cellis, J. E., ed. (1994); “Current Protocols in Immunology” Volumes I-III Coligan J. E., ed. (1994); Stites et al. (eds), “Basic and Clinical Immunology” (8th Edition), Appleton & Lange, Norwalk, Conn. (1994); Mishell and Shiigi (eds), “Selected Methods in Cellular Immunology”, W. H. Freeman and Co., New York (1980); available immunoassays are extensively described in the patent and scientific literature, see, for example, U.S. Pat. Nos. 3,791,932; 3,839,153; 3,850,752; 3,850,578; 3,853,987; 3,867,517; 3,879,262; 3,901,654; 3,935,074; 3,984,533; 3,996,345; 4,034,074; 4,098,876; 4,879,219; 5,011,771 and 5,281,521; “Oligonucleotide Synthesis” Gait, M. J., ed. (1984); “Nucleic Acid Hybridization” Hames, B. D., and Higgins S. J., eds. (1985); “Transcription and Translation” Hames, B. D., and Higgins S. J., Eds. (1984); “Animal Cell Culture” Freshney, R. I., ed. (1986); “Immobilized Cells and Enzymes” IRL Press, (1986); “A Practical Guide to Molecular Cloning” Perbal, B., (1984) and “Methods in Enzymology” Vol. 1-317, Academic Press; “PCR Protocols: A Guide To Methods And Applications”, Academic Press, San Diego, Calif. (1990); Marshak et al., “Strategies for Protein Purification and Characterization—A Laboratory Course Manual” CSHL Press (1996); all of which are incorporated by reference as if fully set forth herein. Other general references are provided throughout this document. The procedures therein are believed to be well known in the art and are provided for the convenience of the reader. All the information contained therein is incorporated herein by reference.

### Example 1

### Screening for Toxicant Responsive Bacterial Promoters Materials and Experimental Procedures

Promoter Libraries

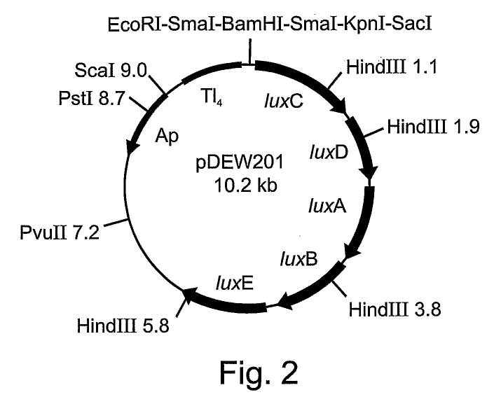

Bacteria and plasmids—The E. coli luxCDABE promoter fusion library was obtained from Dr. Tina Van Dyk of the DuPont Company (Wilmington, Del., USA). This library includes 689 different promoters ligated upstream to the luxCDABE (of Photorhabdus luminescens) inserted into pDEW201 plasmid and transduced into E. coli strain DPD1675 (see FIG. 2). 27% of the E. coli genome is represented in this library including most of the stress response systems [VanDyk, T. K. (2002) Lighting the way: Genome—Wide, Bioluminescent Gene Expression Analysis. ASM News 68:222-230].

An additional strain containing the grpE promoter upstream to the luxCDABE operon of Vibrio Fisheri was tested at 30° C. [VanDyk, T. K., W. R. Majarian, K. B. Konstantinov, R. M. Young, P. S. Dhujati, and R. A. LaRossa. (1994) Appl. Environ. Microbiol 60:1414-1420].

Bacteria storage—The library was kept in 96-well microtiter plates (Costar. USA), at −80° C. (Revco Legaci™. USA) in 20% glycerol solution (Frutarom LTD. Israel). The library was routinely restored every three months by growing each strain overnight in LB with 100 μg ampicillin/ml (Sigma. USA) in a 96-well 2 ml microtiter plate (Costar. USA), at 37° C., 200 rpm (YIH DER LM-530 incubator), followed by re-suspension in glycerol to a final concentration of 20%.

Toxicants—The below listed toxicants were purchased from Sigma USA.

Bacterial cell growth and exposure to toxicants—Each bacterial strain was grown for 20 hours in 1 ml LB including ampicillin (100 μg/ml, Sigma. USA) followed by ×100 dilution into a final volume of 1 ml LB for two hours in a 96-well 2 ml microtiter plate (Costar. USA), at 37° C. 200 rpm (YIH DER LM-530 incubator). Bacteria were then transferred into a 384-well microtiter plate (Costar. USA) containing double dilutions of the toxicant in LB in such a manner that each strain was exposed to seven different concentrations of the toxicant as well as to a control conditions containing LB only.

Luminescence measurement and data analysis—Luminescence was measured during 4-6 hours in 1-1.5 hour intervals using VICTOR² Wallac 1420 luminometer (Turka, Finland). Luminescence is represented by Relative Luminescence Units (RLU). The responses were characterized using several parameters:
A. Luminescence, (RLU).
B. Response Ratio (RR_{L}) was calculated as the ratio between the luminescence measured from the induced well (RLUᵢ) divided by the luminescence measured from the control well (RLU_{c}):

RR_{L}=RLUᵢ/RLU_{c}
C. RRₘₐₓ—Maximal RR_{L} obtained during the course of exposure.
D. Cₘₐₓ—toxicant concentration that produced the RRₘₐₓ.
RRₘₐₓ distribution analysis was effected amongst the whole luxCDABE library to examine the screening procedure s efficiency by differentiating responding from non-responding promoters.

Promoters were divided according to their RRₘₐₓ into the following groups:
A. Non-responsive—0<RRₘₐₓ<2
B. Low-responsive—2<RRₘₐₓ<3
C. Medium-responsive—3<RRₘₐₓ<5
D. Highly-responsive—5<RRₘₐₓ<10
E. Very highly-responsive—10<RRₘₐₓ

RRₘₐₓ distribution that exhibit a high proportion of responding promoters implies that the screening protocol, does not answer the screening objectives. However, a high non-responding combined with a low very high responding proportions implies on a good screening procedure.

Luminescence measurements and data analysis—Luminescence was measured for 2 hours in 4.8 min intervals using VICTOR² Wallac luminometer (Turku, Finland). Average luminescence from the 40 repetitions and standard deviations were calculated using Microsoft Excel.

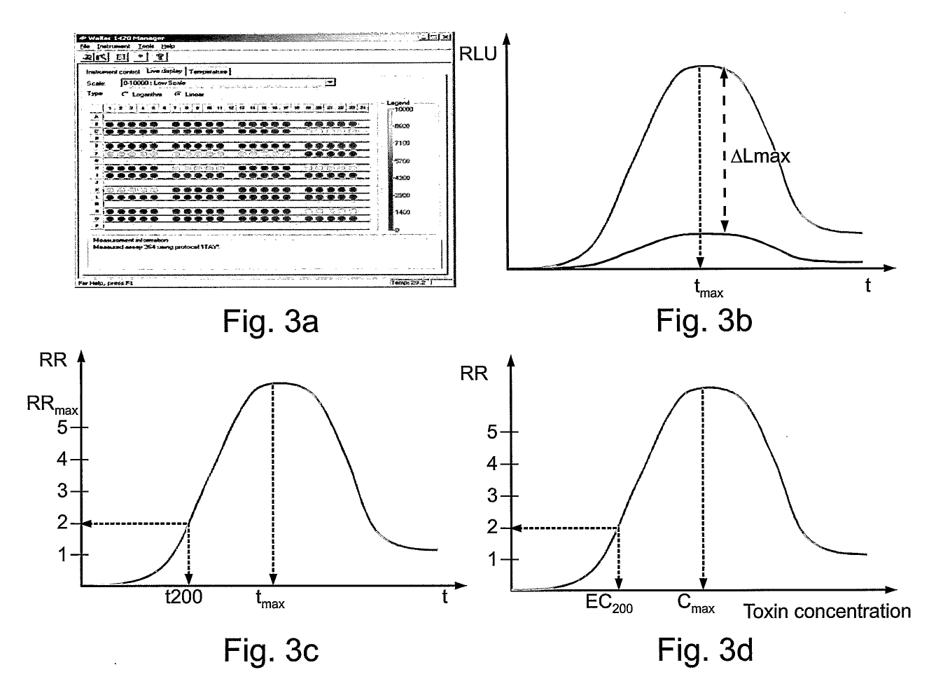

The parameters which were calculated are listed below and illustrated in FIGS. 3b-d. These were selected in order to examine the responses of each strain as well as the whole panel function ability. The selected parameters provide information on the response intensity (RRₘₐₓ, ΔLₘₐₓ) as well as sensitivity (Cₘₐₓ, EC₂₀₀) and response rate (tₘₐₓ, t₂₀₀).
1. RRₘₐₓ—Maximal Response Ratio=RLUᵢ/RLU_{c};
2. ΔLₘₐₓ[RLU]—Maximal Response Differential=RLUᵢ−RLU_{c};
3. Cₘₐₓ[mg/l]—Toxicant Concentration at ΔLₘₐₓ and RRₘₐₓ;
4. tₘₐₓ[min]—Time to reach RRₘₐₓ and ΔLₘₐₓ;
5. EC₂₀₀ [mg/l]—Toxicant concentration at (RLUᵢ−RLU_{c})/RLU_{c}=1 (using Cₘₐₓ);
6. t₂₀₀ [min]—Time at (RLUᵢ−RLU_{c})/RLU_{c}=1 (using Cₘₐₓ);
7. TDC—Target Detection Concentration [mg/l]—LD₅₀(mg/kg)×70 kg/2 L; where LD₅₀ refers to oral exposure in rats.

Results

In order to identify promoters which activity may be induced by toxicants a promoter library including the luciferase reporter gene expressed in E. coli was used. Each bacterial strain was grown for 20 hours in 1 ml LB and diluted into a 96-well 2 ml microtiter plate (Costar. USA). Bacteria were then transferred into a 384-well microtiter including the toxicants listed in Table 1 and 2 below, such that each strain was exposed to seven different concentrations of the toxicant as well as to control conditions consisting of LB only. Toxicants were chosen according to their toxicity and other characteristics unique to their group.

TABLE 1

| | Molecular | | LD₅₀ |
|---|---|---|---|
| Toxicant | formula | Mechanism of action | (mg/kg) |
| Ethyl Parathion | C₁₀H₁₄NO₅PS | AChE Inhibition | 2 |
| Dichlorvos | C₄H₇Cl₂O₄P | AChE Inhibition | 56 |
| Potassium | | Reacts with Iron in | 6.5 |
| Cyanide | | Mitochondrial | |
| | | Cytochrome Oxidase | |
| Nitrogen | C₅H₁₁Cl₂N | Alkylation by Beta-Carbons | 10 |
| Mustard | | c(DNA cross-linking and | |
| | | leavage) | |
| Paraquat | C₁₂H₁₄N₂ | Depletion of cellular NADPH | 150 |
| Methomyl | C₅H₁₀N₂O₂S | AChE Inhibition | 17 |
| Botulinum | | Irreversible binding to | 0.1 |
| Toxicant A | | motorneuron junction | |
| | | (presynaptic) | |

LD₅₀ values were calculated for an oral exposure to rats according to [Toxnet.com, posting date. (Online)].

TABLE 2

| Toxicant | Manufacturer |
|---|---|
| 2 Chlorophenol | Sigma. USA |
| 2,6 Dichlorophenol | Sigma. USA |
| 2,4,5 Trichlorophenol | Sigma. USA |
| Saxitoxicant | Sigma. USA |
| Tetrodotoxicant | Sigma. USA |
| Cholchicin | Sigma. USA |
| Phosdrin | Sigma. USA |

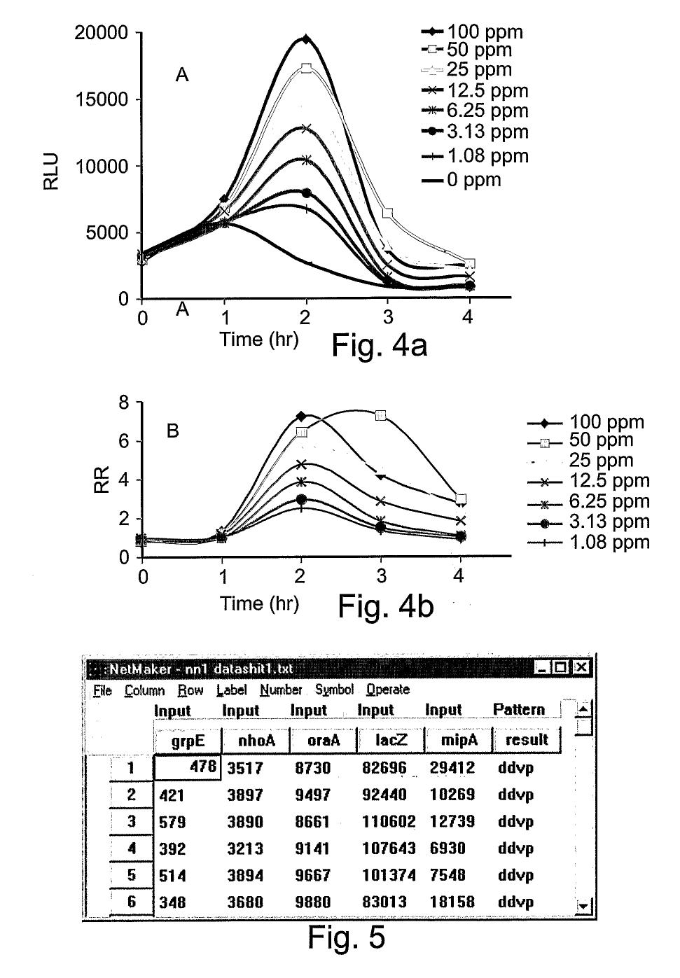

Throughout the screening, response ratios as well as the absolute RLU values were analyzed for: each promoter against all toxicants. Measurements were effected during 4-6 hours every 1-1.5 hour (see FIG. 4a) . High responses due to technical issues (e.g., a sudden drop in the control RLU) were ignored. The distribution of the responses among the library members is summarized in Table 3 below. Promoters which produced the highest response ratios, were isolated and retested. These promoters are listed in Table 4 below, as well as their RRₘₐₓ and Cₘₐₓ values.

TABLE 3

| Toxicant | 0 < RRₘₐₓ < 2 | 2 < RRₘₐₓ < 3 | 3 < RRₘₐₓ < 5 | 5 < RRₘₐₓ < 10 | 10 < RRₘₐₓ |
|---|---|---|---|---|---|
| Ethyl Parathion | 89.6% | 6.3% | 2.1% | 1.7% | 0.4% |
| DDVP | 91.7% | 5.7% | 2.0% | 0.4% | 0.1% |
| Potassium Cyanide | 62.9% | 24.3% | 9.4% | 2.7% | 0.7% |
| Nitrogen Mustard | 84.5% | 9.5% | 4.1% | 1.6% | 0.3% |
| Paraquat | 71.1% | 13.7% | 7.5% | 3.7% | 4.0% |
| Methomyl | 90.1% | 7.1% | 2.0% | 0.7% | 0.1% |
| Botulinum Toxin A | 100% | — | — | — | — |
| Average | 81.7% | 11.1% | 4.5% | 1.8% | 0.9% |

RRₘₐₓ distribution amongst the E. coli luxCDABE library. E. coli strains harboring 679 different promoters were exposed to seven model toxicants in seven concentration. Response ratio distribution was analyzed in order to examine the screening procedure's efficiency. High responses were induced by paraquat. Potassium cyanide, ethyl parathion, nitrogen mustard and DDVP induced lower but detectable responses. Exposure to methomyl produced verylow responses. B-tox exposure did not produce any responses. Average values do not include the B-tox.

TABLE 4

| | | | Cₘₐₓ | | | | Cₘₐₓ |
|---|---|---|---|---|---|---|---|
| Toxicant | Promoter | RRₘₐₓ | (mg/l) | Toxicant | Promoter | RRₘₐₓ | (mg/l) |
| Ethyl Parathion | MipA | 20 | 500 | Nitrogen | oraA | 6 | 31 |
| | | | | Mustard | | | |
| | LacAYZ | 8 | 500 | | recA | 6 | 250 |
| DDVP | GrpE | 7 | 125 | Paraquat | nhoA | 34 | 500 |
| | Fiu | 5 | 500 | | lacAYZ | 28 | 500 |
| Potassium | LacAYZ | 19 | 20 | Methomyl | otsAB | 3 | 2000 |
| Cyanide | | | | | | | |
| | MalPQ | 9 | 20 | | yciD | 2 | 2000 |

Promoters which produced the highest response ratios during the screening. From that list of promoters, six were selected for the biosensor panel. These were: lacZ, grpE, fiu, nhoA, oraA, and mipA.

RRₘₐₓ distribution—Analysis of the RRₘₐₓ distribution amongst the whole library was effected and presented in the table below. The results show that most of the promoters did not respond to toxicants and a small number of promoters produced a high response ratio. Paraquat was found to be the strongest inducer, to which 4% of the promoters produced a response ratio higher than 10. Potassium cyanide, ethyl parathion, nitrogen mustard and DDVP induced less than 1% of the promoters to RRₘₐₓ higher than 10, though a moderate number of promoters produced detectable responses to these toxicants. Exposure to methomyl produced very poor responses from a very limited number of promoters. Retesting of these promoters as well as examination of lower and higher methomyl concentrations showed even poorer results. Exposure to Botulinum Toxin A (B-tox) did not produce any response thus no continued use of this toxicant was made in this research.

Promoter selection—The library screening aim was to select a limited number of bacterial strains, which could respond to a wide variety of toxicants. Following the screening the selected promoters were isolated and validated. The next step in the selection process was cross examining responses of reactive promoters to different toxicants to ensure that the biosensor panel covers as wide a toxicant range as possible.

At the end of this process six promoters were selected for the panel including lacZ, grpE, fiu nhoA, oraA, and mipA.

Table 5 below, shows significant responses of the selected promoters to the different toxicants. As is shown, several promoters responded to the same toxicant, such as nhoA, lacZ, and fiu to paraquat, or grpE, lacZ, and fiu to potassium cyanide. However, to some of the toxicants only one member of the panel produced a response (DDVP, Phosdrin and Cholchicin). Furthermore the characterization results indicated that different response patterns were produced by the panel members in response to the different toxicants.

TABLE 5

| Toxicant | Promoter | RRₘₐₓ | ΔLₘₐₓ | Cₘₐₓ(mg/l) | tₘₐₓ(min) | TDC(mg/l) | EC₂₀₀(mg/L) | t₂₀₀(min) |
|---|---|---|---|---|---|---|---|---|
| Model toxicants | | | | | | | | |
| Paraquat | nhoA | 50.2 | 146,342 | 62.5 | 96/110 | 5,250 | 0.43 | 21 |
| | lacZ | 13.0 | 253,421 | 32.5 | 123/132 | 5,250 | 9.0 | 85 |
| | fiu | 16.9 | 29,591 | 250 | 120/60 | 5,250 | 9.8 | 42 |
| Potassium | grpE | 15.4 | 60,587 | 20 | 96/78 | 230 | 1.0 | 26 |
| Cyanide | | | | | | | | |
| | lacZ | 13.8 | 43,108 | 20 | 100/132 | 230 | 4.0 | 70 |
| | fiu | 5.2 | 7,509 | 20 | 120/120 | 230 | 0.2 | 85 |
| E-Parathion | lacZ | 4.7 | 47,776 | 125 | 132/132 | 70 | 13.0 | 120 |
| | mipA | 20.2 | 196,600 | 500 | 87/110 | 70 | 79.0 | 60 |
| DDVP | grpE | 5.9 | 5,290 | 125 | 132/132 | 1,960 | 32.5 | 41 |
| N-Mustard | oraA | 7.8 | 90,770 | 125 | 110/110 | 350 | 6.1 | 52 |
| | fiu | 4.6 | 7,258 | 125 | 120/120 | 350 | 11.0 | 80 |
| Additional | | | | | | | | |
| toxicants | | | | | | | | |
| 2,4,5- | grpE | 22.1 | 54,432 | 31 | 90/90 | 2,960 | 1.0 | 21 |
| Trichlorophenol | | | | | | | | |
| | lacZ | 16.1 | 74,832 | 31 | 120/120 | 2,960 | 0.8 | 95 |
| | fiu | 11.7 | 33,697 | 31 | 30/120 | 2,960 | 1.2 | 39 |
| 2,6- | grpE | 19.5 | 47,877 | 31 | 90/90 | 6,550 | 1.2 | 29 |
| Dichlorophenol | | | | | | | | |
| | fiu | 4.4 | 25,553 | 125 | 30/120 | 6,550 | 16.0 | 31 |
| 2-Chlorophenol | grpE | 18.7 | 33,345 | 250 | 90/90 | 12,110 | 2.8 | 32 |
| | fiu | 5.8 | 11,971 | 500 | 90/120 | 12,110 | 3.4 | 80 |
| Colchicine | fiu | 4.0 | 1,924 | 45 | 120/120 | 203 | 14.3 | 85 |
| Phosdrin | lacZ | 3.7 | 12,900 | 180 | 120/120 | 170 | 38.3 | 110 |
| Metham Sodium | lacZ | 6.5 | 30,468 | 180 | 120/120 | 28,700 | 20.0 | 90 |
| | mipA | 5.9 | 27,745 | 180 | 120/120 | 28,700 | 18.0 | 100 |

Characterization summary of the responses of the 6 promoters selected for the biosensor panel based on the 40 repetitions experiments. The tested toxicants were paraqut, potassium cayanide, the three organophosphates (ethyl paration, DDVP and phosdrin), the carbamate metham sodium and the chemical warfare agent nitrogen mustard. Responses were characterized according to the maximal response ratio and differential (RRₘₐₓ and ΔLₘₐₓ respectively), theconcentration and time in which the former values were obtained (Cₘₐₓ and tₘₐₓ respectively), the theoretical toxicant concentration and response duration needed to induce a two fold response ratio (EC₂₀₀ and t₂₀₀ respectively).

### Example 2

### Toxicant Recognition Using Neuronal Networks

Identification of the pattern of response of the bacterial strains in the panel provides information on the identity of the toxicant in the sample. A commercially available neural network (nn) software was used to effect this analysis. The human brain cells perform intelligent operations and can be trained by repeated presentation of examples, which include both inputs and outputs. The neural network software operates based on this notion. For example, in the neural network software of the present invention the input is the reporting system's signal intensity (e.g. luminescence, fluorescence) while the output is the toxicant identity. Nns, just like human beings, learn by example and repetition. At a fundamental level, all nns learn associations. When the network sees particular input data, it responds with particular output data. The software is based on a large-scale statistical analysis of the input data, which is amended following each training session to the desired accuracy [Dvorak, J. (1991) Best of 1990: BrainMaker Professional, Version 1.5. PC Magazine]. When the nn is being trained, it is presented with many input-output pairs of data. These input-output pairs are called “facts”. Following training the nn can be tested with data that was not introduced to it before and only then the nn can run with real data.

Every responsive promoter produces a unique response pattern to different toxicants. These fingerprints can be characterized in three dimensions (i.e., intensity, time and toxicant concentration). Ones the nn identifies the response pattern of each promoter to each toxicant it can detect the toxicant present in the sample according to the panel's response.

Typically the formation of nns includes a number of steps: First the is edited in the appropriate way to be introduced into the nn. Then the nn is trained and tested before being used with unknown data.

Experimental Procedures

To design the nn for toxicant detection, data collected from the characterization experiments was used. Specifically used were the RLU values and not with other parameters such as response ratio etc. since it is expected that the network will analyze the data by itself according to its mathematical functions. Any manipulation on the data could interfere with the data processing by the nn. The formation of the nn was effected as follows.

Data Preparation

RLU values produced by five members of the biosensor panel (i.e., grpE, lacZ, mipA, oraA, and nhoA) in response to sterile LB and the five model toxicants, which have elicited adequate responses (i.e., DDVP, ethyl parathion, paraquat, potassium cyanide, and nitrogen mustard) were used as input values. The input values were based on the RLU values measured from the panel after 30, 60, and 120 minutes as response to the Cₘₐₓ of the different toxicants. At this stage data was simplified by ignoring the time dimension as well as the toxicant concentration. Thus three separate nn systems were generated for 30, 60, and 120 minutes. The output entered was the name of the toxicant, i.e. “DDVP” (see FIG. 5).

The data was arranged in a table and introduced to the NetMaker software (BrainMaker, California Scientific Software) as illustrated in FIG. 5. Using this software data was gathered and training as well as testing files were generated. In total, 240 “facts” were entered into the NetMaker file for each nn system.

A fourth nn system based on the same data source was set as well. However, in this system data collected during the first 30 minutes (8 RLU readings) was used, thus taking into account the time dimension. This system will be referred to as “time course nn”.

Training the Neuronal Network—Following preparation, data was exported to the nn software for training. During training the nn was presented with the input as well as output data, which was repeatedly entered until the nn output matched the entered data output of all facts or until it was stopped. Note, each cycle is termed a “run”. The training of the nn was stopped after more than 215,000 runs, followed by testing the nn. Training of the time course system was completed after about 5 seconds and was stopped automatically by the software.

Testing the Neuronal Network—Typically (i.e., default), while creating the data files the nn creates a test file, which contains 10% of the entered facts. These are “hidden” from the nn during the training step and are used only for the purpose of testing the nn. Testing was done in a single step mode. Thus, of the 240 facts entered, 24 were used for the testing when each one was tested individually. The output produced by the nn got a value on a scale between 0 and 1 when 1 represents 100% probability for the toxicant's presence in the sample. This value is referred to as detection probability or DP.

Running the Neuronal Network—This is the final and most important step of the process and its results are detailed in the Results section below. Although the nn tests itself using unknown data, another data source was used for testing the network. Basically, this data included average RLU values of the 40 repetitions of each panel member 30, 60 and 120 minutes after toxicant exposure. The networks were also introduced to RLU values collected from the panel in response to lower toxicant concentrations, in order to test their ability to identify low toxicant concentrations.

Two more tests were carried out in order to examine the nn performance. The first was with data collected from the freeze-dried bacteria in response to paraquat exposure the second was with data collected from the panel in response to methomyl and 2,4,5 trichlorophenol. Both sets of data were gathered from experiments which took place in a 96-well microtiter plate while the nn training data was collected from experiments in a 384-well plates. Running of the time course system was carried out using the average RLU values collected during the first 8 RLU readings (30 minutes).

Results:

Toxicant Identification

Self-testing results—As mentioned above the nn tests itself with 10% of the data used for the training Thus the four nn systems were tested with 24 facts producing the following results:
(i) 120 minutes nn: 24 of the 24 facts were correctly analyzed.
(ii) 60 minutes nn: 19 of the 24 facts were correctly analyzed (iii) 30 minutes nn: 22 of the 24 facts were correctly analyzed.
(iv) Time course nn: 24 of the 24 facts were correctly analyzed.

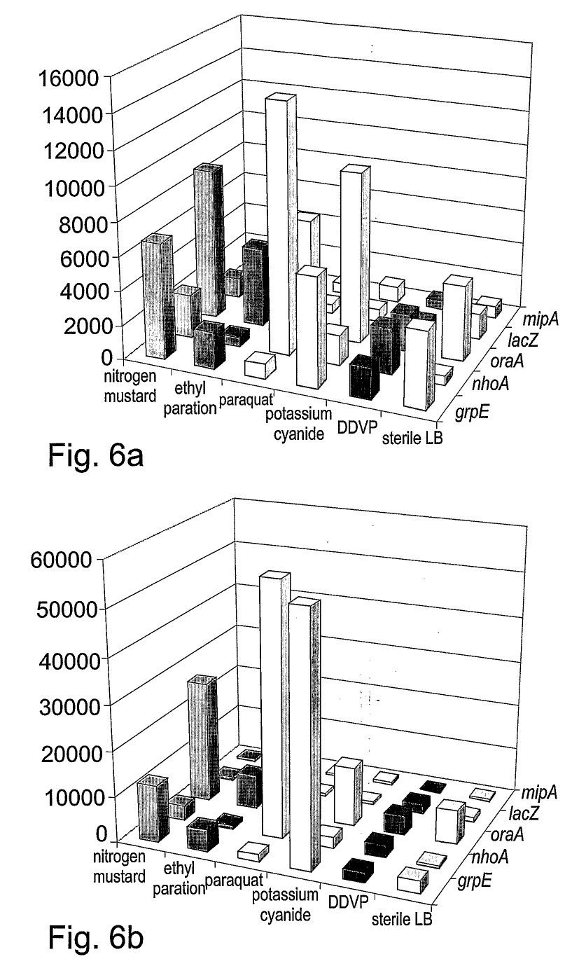

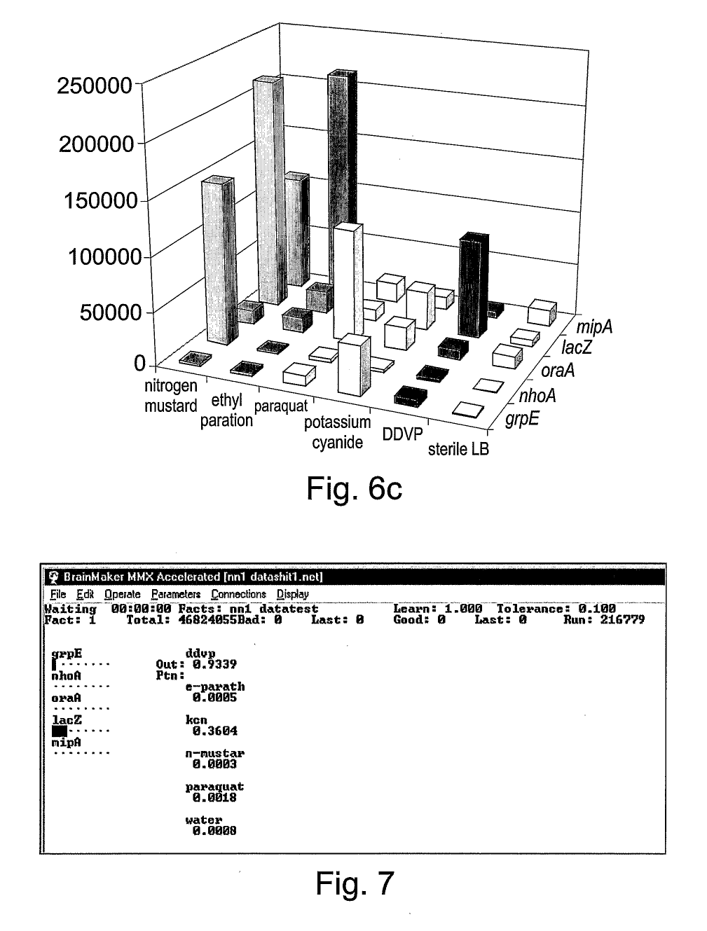

Running the nn—As described above the nn was introduced with average RLU values collected from 40 repetitions of the panel members in response to five different toxicants and sterile LB at a given time (FIG. 6). The resulting DP values are detailed in Table 6 below and illustrated in the BrainMaker software format in FIG. 7. In Table 6 the left column represents the actual toxicant present in the sample while the top row represents the output values produced by the nn as a result of the data introduction to the network. The right column represents the false negative values. These are the probabilities that a poisoned sample is considered a safe one. In the same way the bottom row represents the false positive values, indicating the probability of a safe sample to be poisoned.

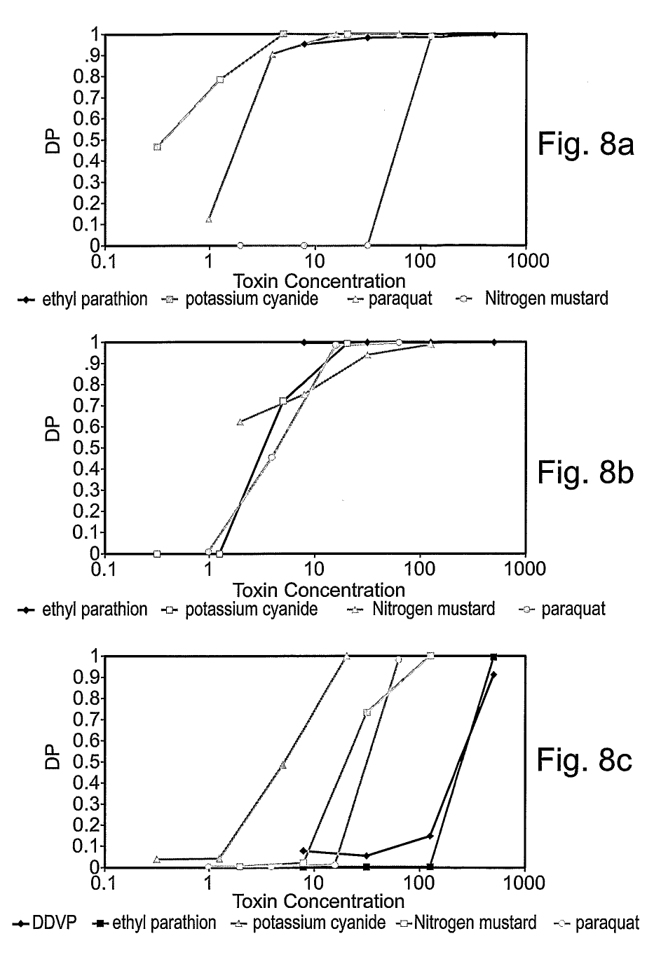

FIGS. 8a-c show the RLU values used for the running the three nn systems. The nn was introduced with average RLU values collected from 40 repetitions of the panel members in response to five different toxicants and sterile LB 30, 60, and 120 minutes following exposure (FIGS. 8a, 8b, and 8c respectively).

As is evident from Table 6 the 30 minute nn was able to successfully identify 5 of the six tested materials but failed to detect the DDVP. However, it did not respond to it as if it were a safe sample. The DP values for the detected compounds were higher than 99%. The false positive rate did not exceed 0.0064 and was usually very low. The false negative rate was low as well ranging from 0.0003 to 0.0362 (the latter value was given to the DDVP sample).

From the 60 minutes nn output it seems that the nn was able to identify most of the toxicants. Nevertheless the nn misjudged the DDVP to be sterile LB. That was probably due to the fact that the response of the panel to DDVP after 60 minutes is not significant enough. This will be discussed further hereinbelow. No similarities were found between the two organo phosphates. The false positive rate was generally low though the safe sample had 29% probability to be ethyl parathion. Other than that the nn was able to detect the sterile LB as a safe sample. The false negative rate was usually very low (between 0.0003 to 0.0032) yet the DDVP was mistaken to be a safe sample.

Looking, at the 120 minute nn outputs, it is obvious that the nn could detect with a very high accuracy the different toxicants. The lowest DP values was 0.9339 for the detection of DDVP while others were higher than 0.98. The false positive rate ranges from 0.0003 to 0.0113 indicating that the nn false alarm rate will be potentially very low. More importantly the false negative rate ranges from 0.0003 to 0.0008. Any similarity between the two organo phosphates was not found though the DP for DDVP when ethyl parathion was actually present, was relatively high (0.0884). This may be due to the limited number of toxicants, which the nn was trained to detect. Another observation is that the distinction of potassium cyanide is relatively poor. The DP for potassium cyanide when DDVP was present was 0.3604 however the reciprocal value was 0.0003 indicating that the nn does not distinct well potassium cyanide from DDVP.

TABLE 6

| Output | | | Potassium | Nitrogen | | Sterile |
|---|---|---|---|---|---|---|
| Actual | DDVP | Ethyl Parathion | Cyanide | Mustard | Paraquat | LB |
| 30 min nn | | | | | | |
| DDVP | 0.0003 | 0.3269 | 0.0010 | 0.0003 | 0.0042 | 0.0362 |
| Ethyl Parathion | 0.0003 | 0.9939 | 0.0008 | 0.0003 | 0.0022 | 0.0013 |
| Potassium | 0.0020 | 0.0015 | 0.9996 | 0.0005 | 0.0003 | 0.0120 |
| Cyanide | | | | | | |
| Nitrogen | 0.0003 | 0.0003 | 0.0191 | 0.9900 | 0.0069 | 0.0013 |
| Mustard | | | | | | |
| Paraquat | 0.0003 | 0.0003 | 0.0003 | 0.0005 | 0.9976 | 0.0003 |
| Sterile LB | 0.0003 | 0.0003 | 0.0064 | 0.0025 | 0.0003 | 0.9974 |
| 60 min nn | | | | | | |
| DDVP | 0.0406 | 0.0003 | 0.0005 | 0.0003 | 0.0003 | 0.9932 |
| Ethyl Parathion | 0.0003 | 0.9993 | 0.0557 | 0.2342 | 0.0027 | 0.0003 |
| Potassium | 0.0284 | 0.0005 | 0.9944 | 0.0132 | 0.0003 | 0.0032 |
| Cyanide | | | | | | |
| Nitrogen | 0.0044 | 0.0003 | 0.0039 | 0.9886 | 0.0047 | 0.0003 |
| Mustard | | | | | | |
| Paraquat | 0.0005 | 0.0003 | 0.0003 | 0.0047 | 0.9983 | 0.0003 |
| Sterile LB | 0.0008 | 0.2906 | 0.0152 | 0.0003 | 0.0003 | 0.9950 |
| 120 min nn | | | | | | |
| DDVP | 0.9339 | 0.0005 | 0.3604 | 0.0003 | 0.0018 | 0.0008 |
| Ethyl Parathion | 0.0884 | 0.9961 | 0.0088 | 0.0003 | 0.0003 | 0.0003 |
| Potassium | 0.0003 | 0.0003 | 0.9998 | 0.0003 | 0.0003 | 0.0003 |
| Cyanide | | | | | | |
| Nitrogen | 0.0003 | 0.0003 | 0.1829 | 0.9998 | 0.0003 | 0.0003 |
| Mustard | | | | | | |
| Paraquat | 0.0301 | 0.0003 | 0.0003 | 0.0003 | 0.9834 | 0.0003 |
| Sterile LB | 0.0025 | 0.0003 | 0.0113 | 0.0005 | 0.0013 | 0.9971 |

The output values produced by the three Neuronal Networks. The values describe the probability (on a 0 to 1 scale) for the presence of the suspected toxicant in the sample based on the RLU values entered. The rows represent the actual toxicant in the sample while the columns represent the output values produced by the network. The table includes the output data by the 30, 60 and 120 minutes neuronal networks. The bottom row of each neuronal network, describing the outputof sterile LB actually represents the false positive probability let alone the last column. On the other hand the last column actually represents the false negative probability.

Responses of the network to different toxicant concentrations—The first step of running the nn was with data collected as a response to the same toxicant concentration as the nn was trained with. The nn was tested with RLU values collected from the panel in response to lower toxicant concentrations. The nn outputs are illustrated in FIGS. 8a-c representing the 30, 60 and 120 minutes nns respectively. The graphs describe the nn DP values versus the toxicant concentration. As shown the DP dependence on toxicants concentrations decreases with time. In, other words, innetworks, which were trained with “late” data detection of low toxicant concentrations was difficult. In general the only toxicant that was detected at low concentration was ethyl parathion (only in the 30 and 60 minutes systems). The systems were notable to detect any other low concentration of toxicants.

Running of the time course nn system—The neural network system of the present invention was tested with data collected from the panel in response to the same toxicant concentration as the 30, 60, and 120 minutes systems. The output DP values are listed in Table 7, below. The results indicate that the system can already detect after 30 minutes low concentrations of DDVP, ethyl parathion, potassium cyanide, and paraquat. The system failed to detect low nitrogen mustard concentrations, but was able to identify it at Cₘₐₓ.

The system successfully identified the sterile LB with a DP value of 0.9986 and a false positive rate of 0.0003 to 0.0049. The false negative rate ranged from 0.0003 to 0.0108 (in response to potassium cyanide).

TABLE 7

| Toxicant | | | | | |
|---|---|---|---|---|---|
| dilution | DDVP | ethyl parathion | potassium cyanide | nitrogen mustard | paraquat |
| 1 | 0.9900 | 0.9976 | 0.9937 | 0.9605 | 0.9964 |
| ¼ | 0.9842 | 0.9956 | 0.9932 | 0.2615 | 0.9961 |
| 1/16 | 0.9812 | 0.9954 | 0.9661 | 0.0225 | 0.9114 |
| 1/64 | 0.9803 | 0.9944 | 0.9427 | 0.0130 | 0.0533 |

DP values produced by the time course system. The nn was tested with data collected from the panel in response to low toxicant concentrations. Though the training of the nn was done with data collected in response to the Cₘₐₓ only, the system was able to detect in most cases lower toxicant concentrations after only 30 minutes.

### Example 3

### Drying Bacterial Cells for Long Term Storage

To study the effects of drying an cell short and long-term viability, activity and storage stability two drying protolocols were assessed; a freeze drying protocol and a high temperature drying (HTD) protocol.

### Example 3a

### Freeze Drying

Materials and Experimental Procedures

Freeze-drying protocol—A simple drying protocol, which did not include a secondary drying step was used. The drying formulation consisted of a 12% trehalose solution. Typically, 300 μl aliquots of the bacterial-formulation mix were placed into 8 ml lyophilization vials. The vials were placed on a pre-cooled shelf of a lyophilizer (−40° C.), let to equilibrate and frozen for ca. 90 min followed by vacuum application (ca. 30 mTorr). The drying process was carried out with a shelf temperature of −40° C. for ca. 18 hours. Vials were then capped under vacuum and stored at 37° C. (representative of accelerated aging processes) or −20° C. (representative of a conventional storage temperature).

Results

Viability and activity of freeze-dried bacteria were assessed before and immediately after drying and at pre-determined storage time intervals. The residual moisture of dried samples was checked once and was found to be in the range of 10-12%.

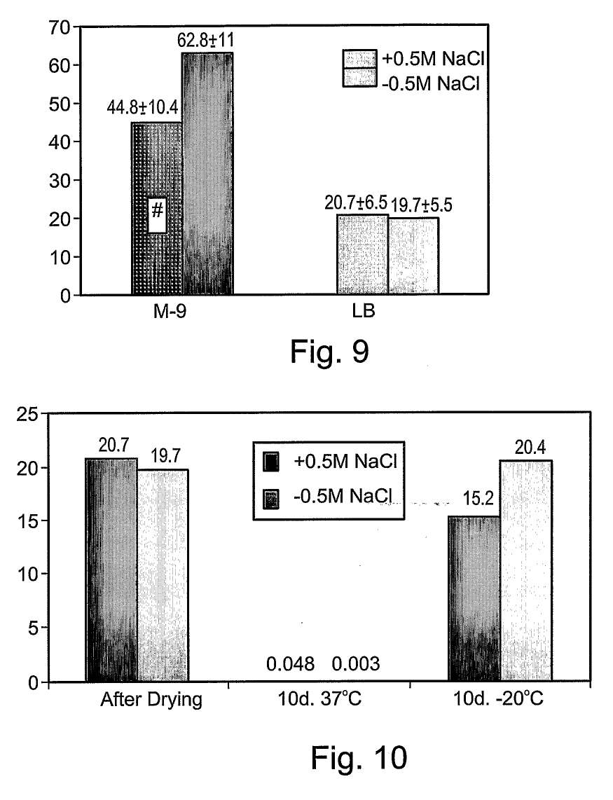

Survival rates immediately after drying (depending on growth media) were 10-60% (see FIG. 9) and survival rates upon storage of ca. 10 days at 37° C. ranged 0.01-0.2% (see FIG. 10). The high range survival rates were obtained with bacteria grown in M-9.

### Example 3b

### High Temperature Drying Protocol

Materials and Experimental Procedures—see U.S. Pat. Nos. 6,468,782, 6,509,146, 6,537,666, 5,766,520, 6,306,345, 5,149,653 and 6,426,210).

High temperature drying protocol—The drying formulation consisted of a 38% trehalose and 1.5% of Polyvinyl-Pyrrolidone (PVP), which served as a viscosity enhancer and, cake strengthener. 200 μl aliquots of the bacterial-formulation mix were placed in 8 ml lyophilization vials. The vials were placed on a pre-warmed shelf of the lyophilizer (+30° C.), let to equilibrate and then vacuum was applied (ca. 30 mTorr). The drying process was carried out with a shelf temperature of +30° C. for ca. 18 hours. Vials were then capped under vacuum and stored at 37° C. or −20° C.

Viability and activity of dried bacteria were assessed at pre-determined time intervals. The residual moisture of dried samples was checked once and the results were in the range of 9-10%.

Results

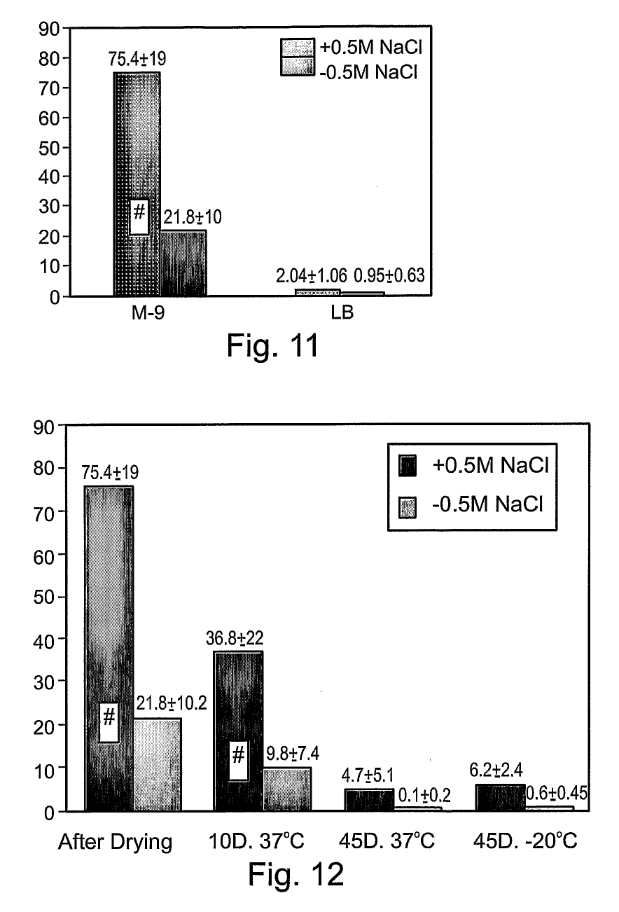

Survival rates immediately after drying (depending on growth media) were 1.0-70% (see FIG. 11) and survival rates upon storage of ca. 10 days at 37° C. ranged 0.5-40% (see FIG. 12). The high-range survival rates were obtained with bacteria grown in M-9 media

Altogether the results presented herein indicate that the HTD protocol achieves better results than the freeze drying protocol in term of cell survival.

### Example 4

### On Chip Lyophilization and Revival

Example 3 above summarize experimental procedures for long term stable storage of bacteria. In order to on-chip lyophilize bacterial cells the present invention attempted for the first time lyophilization of very small volumes (i.e., 0.5-2 μl), once cells are already placed on the chip cavity. Note that in order to simulate drying in a cavity-like volume, most experiments were carried out in 1536-well microtiter plates.

In general the activity of Lux-including strains was tested in final volume of 1 μl or less per well, in 1536-well microtiter plate. Only 1 of 4 wells was used, and activity was measured in Victor² Luminometer, in 384 wells designated program. In most lyophilization experiments 12% Trehalose was used as osmo-cryoprotectant.

Materials and Experimental Procedures

Lyophiliztiion—Single colonies of E. coli strains DPD2794 or TV1061, containing recA::Lux or grpE:Lux plasmid fusions, respectively, were inoculated in liquid LB-Kanamycin medium for overnight growth at 37° C. These cultures were diluted 1:300 in fresh LB medium without antibiotics to a final volume of 50 ml, and allowed to grow at 30° C. to the early logarithmic growth phase at a turbidity of OD₆₀₀ₘₘ=0.1, which is equivalent to 10⁷ cells/ml. The cultures were then concentrated by centrifugation and resuspended to the desired concentration either in 12% trehalose for lyophilization, or in LB for fresh cell induction. Lyophilization, as well as induction, of cell suspensions was effected in opaque white 1536-well microtiter plates (Costar). The microtiter plate containing cell suspensions was lyophilized in an Advantage, Virtis Lyophilizer for overnight under vacuum conditions of 20 mTorr, at −40° C. shelf-temperature. Prior to vacuum application, bacteria were frozen by ramping the shelf temperature from 0° C. to −40° C. at a rate of 0.23° C. min⁻¹. Aliquots of tested compounds diluted in LB were added to the lyophilized bacteria-containing microtiter plate and induction was followed by measuring luminescence in a Wallac Victor² luminometer.

Results

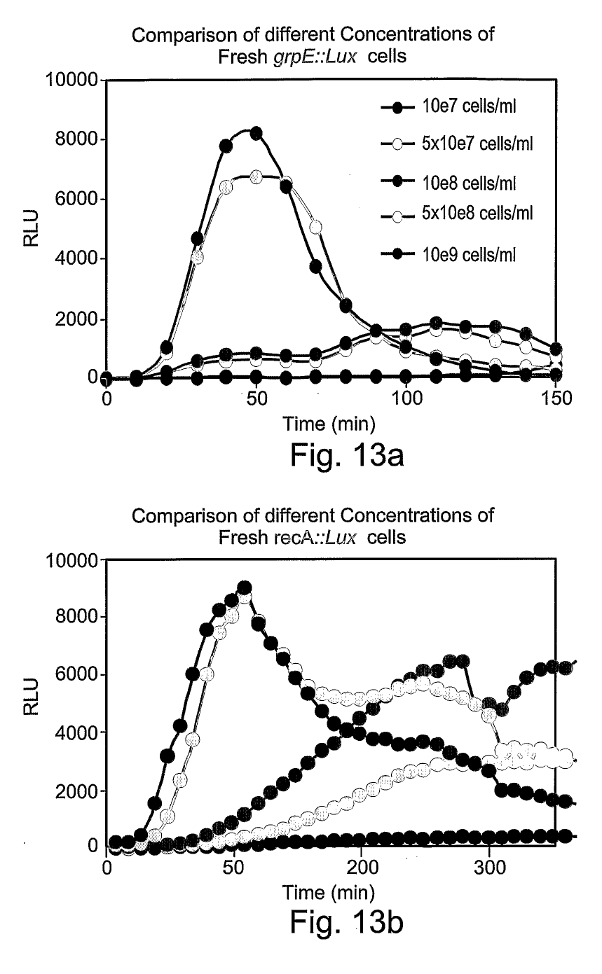

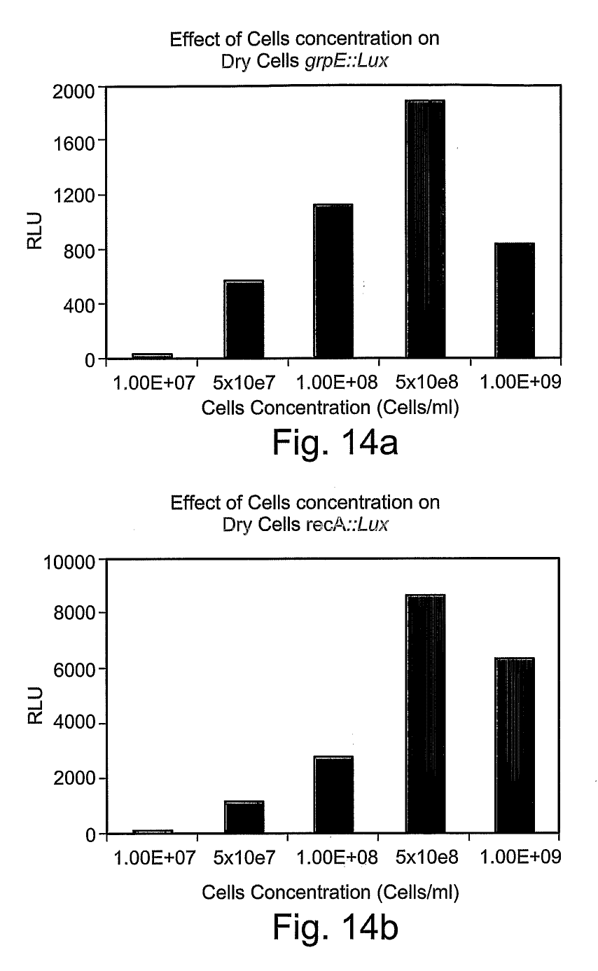

As is shown in FIGS. 13a-b, optimal induction for fresh recA::Lux cells, as well as for grpE::Lux cells, was obtained in cell concentrations of 5×10⁸ and 10⁹ cells/ml. Optimal induction of dry cells of both types was obtained in cell concentrations of 5×10⁸ cells/ml (FIGS. 14a-b). This cell concentration, in a volume of 1 μl, suggests the use of 5×10⁵ cells/well.

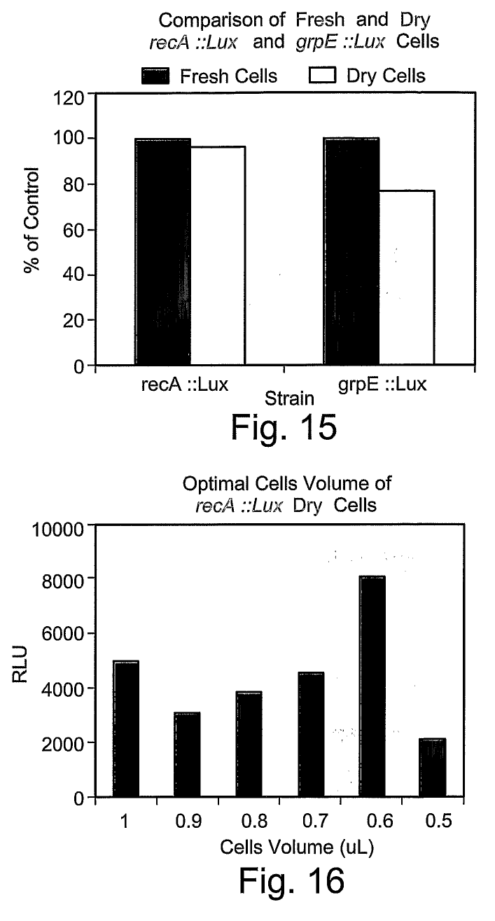

A comparison of the activity of the dry versus the fresh cell revealed that a relatively small fraction of the activity was lost upon drying: 96% and 76% were maintained for the recA::Lux and grpE::Lux cells, respectively (FIGS. 15a-b).

FIG. 16 depicts the results of an experiment in which a single cell concentration was used (5×10⁸ cells/ml), in different volumes. The differences were not high, but it appears that for this cell concentration a volume of 0.6 μL is preferable.

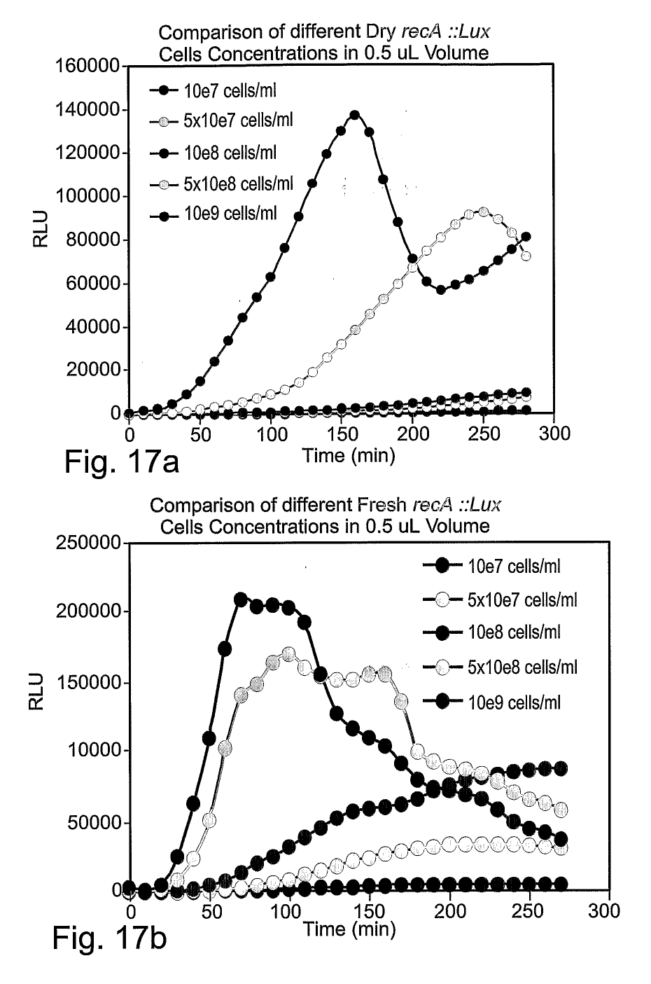

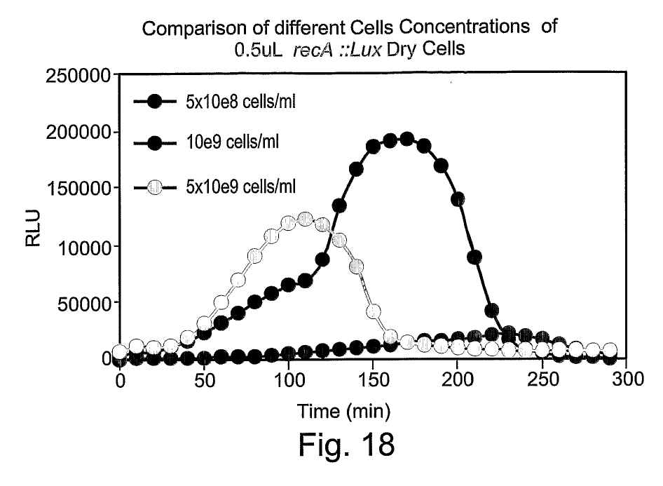

The effects of varying cell concentrations was studies using 0.5 μL as a constant sample volume. As is shown in FIGS. 17a-b, for both fresh and dry recA:;Lux cells, best induction occurred at the highest cell concentration tested, 10⁹ cells/ml (5×10⁵ cells/cavity). As is shown in FIG. 18, a five-fold higher cell density was tested as well, indicating a lowered activity but an earlier signal development.

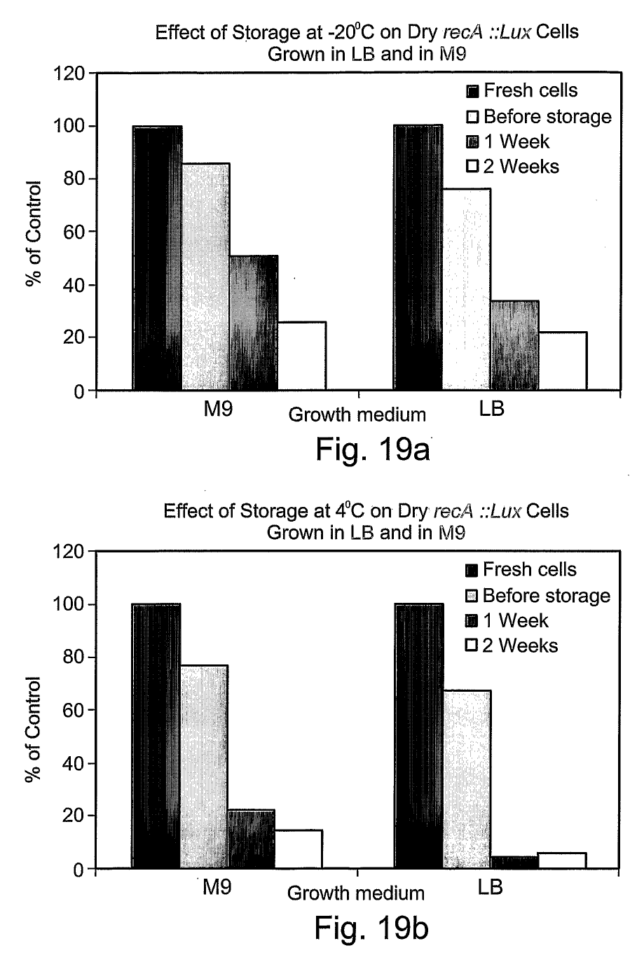

In all the experiments described above, activity data were of cells shortly following the lyophilization procedure. Comparison of residual activity following prolonged storage is depicted in FIGS. 19a-b, for cells pre-grown in rich (LB) or minimal (M9) media. Following 2 weeks of storage, approximately 20% of the activity was retained in cells maintained at −20° C. and 5-15% at 4° C. Growth in M9 appeared to have some beneficial effect.

### Example 5

### Developing and Optimizing a Yeast Cell Population which Responds to a Variety of Toxicants

The baker's yeast Saccharomyces cerevisiae is a key model organism for the study of the physiology of eukaryotic cells (10, 13, 19, 22). Yeast can serve as such a model for two major reasons: i) it is a very simple, easy and cost effective genetically accessible organism; ii) though being a very simple organism, its molecular processes and biochemical pathways are very similar to those of mammalian cells. Because it is so attractive and so relevant as a model organism, a variety of strong technological, experimental and analytical tools have been developed for improving the use of yeast in basic research and biotechnology. As a result S. cerevisiae has become a leading model for biochemistry and molecular biology (1, 2, 3, 5, 6, 7, 8, 13, 17, 18), as well as for genome research, bioinformatic, drug discovery and analysis of drug targets (4, 9, 11, 12, 15, 23, 26). In spite of their great potential and relevancy to human, yeast have not been used as sensors of toxic agents.

The present inventors developed a battery of yeast strains that host a family of sensor genes. Transcription of these sensor genes is induced through cis-acting-stress-responsive elements. Those elements were introduced in different combination upstream to a minimal promoter. About 250 sensor-reporters were engineered this way and were screened for their responsiveness to drugs. Some were nicely responsive. Also constructed and tested were a few sensors driven by full promoters and not by artificial elements. Those were found to be less active. The cis-elements used are normally located at promoters of the yeast stress response.

Experimental Procedures

Promoter selection—The following promoters were used for construction of the yeast panel.

STRE—most stress responsive promoters contain a short sequences (5 base pairs long) known as the Stress Responsive Elements (STREs). STREs bind two transcription factors, Msn2 and Msn4 which are activated in response to many stresses including heat shock, osmotic shock, oxidative stress, and to many types of drugs and metabolic inhibitors.

HSE—a cis-element known as the Heat Shock Element which is found in the promoters of a subset of stress genes, in particular in genes encoding heat shock proteins. HSE is activated following stress by a transcription factor known as the Heat Shock Factor (HSF).

ARE—AP-1 Recognition Element. A cis-element found in the promoters of genes activated primarily by oxidative stress. ARE is a binding site for a transcription factor called yeast AP-1 (yAP-1). iv) GCRE—a binding site for the transcription factor Gcn4. Gcn4 is a bZIP factor, and is a functional homolog of the mammalian c-Jun. It is activated by starvation, UV radiation and metabolic drugs

CRE—homologous to the mammalian cAMP responsive element, which is activated by osmotic shock.

Identification of other toxicant-sensitive promoters cab be effected by screening a combinatorial family of reporters or by analyzing global changes in gene expression following treatment of the cells with the compound.

To significantly increase the sensitivity of the detection system mutants of the stress signaling cascades can be employed. These can be used to produce yeast strains which are highly sensitive to stress (and toxicity). These strains are anticipated to activate the sensors at low concentrations of the toxic compounds.

Results

Promoter based sensors—Promoters of the HSP104, TPX2 and HIS4 genes which are responsive to heat stress, oxidative stress and metabolic inhibitors respectively, were cloned and tested. cis-elements based sensors—the use of large promoter ensures basic transcriptional activity as well as faithful activation of the reporter. Yet, such sensors promoters give rise to high background. They are excellent tools for monitoring: any stress and for continuous verification of the monitoring system (because background signal will be transmitted constantly). Yet they will not be highly specific nor very sensitive. To produce specific and sensitive sensor we shall use the CYC1 mninimal promoter. Stress responsive cis-elements (AREs, HSEs, etc.) will be added upstream to the CYC1 promoter. Such sensors will be highly specific, responsive only to chemicals that activate the given cis-element.

Two sets of artificial sensing genes were constructed: one set with the LacZ reporter gene and the second set with GFP. Below listed are selected examples of the constructs which were made are:

TABLE 8

| Plasmid identifier | Reporter gene |
|---|---|
| (AP1-STRE)1-GFP | GFP |
| (AP1-STRE)2-GFP | GFP |
| (AP1-STRE*3)R-GFP | GFP |
| (AP1-STRE*3)-GFP | GFP |
| (HSE)5-GFP | GFP |
| (HSE)4-GFP | GFP |
| (HSE)(HSE)R-GFP | GFP |
| (AP1-STRE*3)(HSE)R-GFP | GFP |
| (AP1-STRE)1-LacZ | LacZ |
| (AP1-STRE)2-LacZ | LacZ |
| AP1-STRE*3)R-LacZ (R = reverse) | LacZ |
| (AP1-STRE*3)-LacZ | LacZ |
| (HSE)5-LacZ | LacZ |
| (HSE)4-LacZ | LacZ |
| (HSE)HSE)R-LacZ | LacZ |
| (AP1-STRE*3)(HSE)R-LacZ | LacZ |
| AP-1(SV40)*5-LacZ | LacZ |
| AP-1(TRX2)*2-LacZ | LacZ |
| (AP-1-SKN7)-LacZ | LacZ |
| (AP-1_SKN7)R-LacZ | LacZ |
| GCRE*2-LacZ | LacZ |
| GCRE(HIS4)-LacZ | LacZ |
| SKN7-LacZ | LacZ |
| SKN7*5-LacZ | LacZ |

The above-described constructs were transfected and tested for activity in the presence of methomyl and H₂O₂ or Heat shock. This was done in order to choose the most sensitive promoters for further testing against the all list of the model toxicants and in order to see what is the contribution of each element to sensitivity and which combination is the best.

Responsiveness of the above-listed constructs is illustrated in Table 9, below. As is shown the best combination of cis-regulatory elements includes several repeats of the dual element (AP1-STRE). Unexpectedly the HSE element did not contribute to toxins sensitivity, since a construct possessing the HSE element alone even in several repeats responded well to Heat shock but did not respond to toxicants. Accordingly, addition of this element to the responding construct (API-STRE*3)-LacZ did not improve its sensitivity.

Table 9, below lists the responses of the above-described reporter constructs towards parathion methomyl and mustard nitrogen in yeast. Thus, W303-STRE-GFP and W303-(API-STRE)2-GFP responded to parathion rapidly (i.e., a response can be seen within 5 to 10 minutes in very low concentration). A 4-5 fold higher response was observed within an hour.

TABLE 9

| Elements | Responsiveness |
|---|---|
| (AP-1-STRE)*2 | ++++ |
| (AP-1-STRE) | +/− |
| AP-1 | + |
| STRE | ++ |
| −300HSP104 | ++ |
| (HSE)*5 | − |
| AP-1-STRE*3 | ++ |
| AP-1-STRE*3-HSE | ++ |
| SKN7 | − |
| SKN7*5 | − |

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable subcombination.

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims. All publications, patents and patent applications and accession numbers mentioned in this specification are herein incorporated in their entirety by reference into the specification, to the same extent as if each individual publication, patent or patent application was specifically and individually indicated to be incorporated herein by reference. In addition, citation or identification of any reference in this application shall not be construed as an admission that such reference is available as prior art to the present invention.

1-55. (canceled)

56. A population of cells comprising at least two subpopulations of cells, wherein a first subpopulation of said at least two subpopulation of cells includes a first reporter expression construct being expressible in a cell of said first subpopulation when exposed to a first analyte and whereas a second subpopulation of said at least two subpopulation of cells includes a second reporter expression construct being expressible in a cell of said second subpopulation when exposed to a second analyte.

57. The population of cells of claim 56, wherein each of said reporter expression construct includes a cis-acting regulatory element being operably fused to a reporter gene.

58. The population of cells of claim 57, wherein said cis-acting regulatory element is a promoter.

59. The population of cells of claim 58, wherein said promoter is selected from the group consisting of MipA, LacZ, GrpE, Fiu, MalPQ, oraA, nhoA, recA, otsAB and yciD

60. The population of cells of claim 56, wherein each of said first or said second analyte is independently selected from the group consisting of a condition and a substance.

61. The population of cells of claim 56, wherein each of said at least two subpopulations of cells is tagged.

62. A device for detecting presence, absence or level of a substance in a sample, the device comprising a substrate being configured for supporting a population of cells including at least two subpopulations of cells, wherein a first subpopulation of said at least two subpopulations of cells includes a first reporter expression construct being expressible in a cell of said first subpopulation when exposed to a first analyte and whereas a second subpopulation of said at least two subpopulation of cells includes a second reporter expression construct being expressible in a cell of said second subpopulation when exposed to a second analyte, wherein each of said at least two subpopulations of cells is attached to said substrate in an addressable manner.

63. The device of claim 62, wherein each of said reporter expression construct includes a cis-acting regulatory element being operably fused to a reporter gene.

64. The device of claim 63, wherein said cis-acting regulatory element is a promoter.

65. The device of claim 64, wherein said promoter is selected from the group consisting of MipA, LacZ, GrpE, Fiu, MalPQ, oraA, nhoA, recA, otsAB and yciD.

66. A system for detecting presence, absence or level of a substance in a sample, the system comprising:
(a) a device including a substrate being configured for supporting a population of cells including at least two subpopulations of cells, wherein a first subpopulation of said at least two subpopulations of cells includes a first reporter expression construct being expressible in a cell of said first subpopulation when exposed to a first analyte and whereas a second subpopulation of said at least two subpopulation of cells includes a second reporter expression construct being expressible in a cell of said second subpopulation when exposed to a second analyte, wherein each of said at least two subpopulations of cells is attached to said substrate in an addressable manner;
(b) a detector for detecting expression from each of said first and second reporter expression constructs in said population of cells; and
(c) a processing unit for obtaining and processing data representing said expression detected by said detector to thereby provide information relating to the presence, absence or level of the substance in the sample.

67. A method of detecting presence, absence or level of a substance in a sample the method comprising:
(a) exposing a population of cells to the sample, said population of cells including at least two subpopulations of cells, wherein a first subpopulation of said at least two subpopulations of cells includes a first reporter expression construct being expressible in a cell of said first subpopulation when exposed to a first analyte and whereas a second subpopulation of said at least two subpopulation of cells includes a second reporter expression construct being expressible in a cell of said second subpopulation when exposed to a second analyte with the sample; and
(b) analyzing expression of said reporter expression constructs in each of said at least two subpopulations of cells, to thereby detect presence, absence or level of the substance in the sample.

68. The method of claim 67, wherein each of said reporter expression construct includes a cis-acting regulatory element being operably fused to a reporter gene.

69. The method of claim 67, wherein said reporter gene is selected from a group consisting of a fluorescent protein, an enzyme and an affinity tag.

70. The method of claim 68, wherein said cis-acting regulatory element is a promoter

71. The method of claim 70, wherein said promoter is selected from the group consisting of MipA, LacZ, GrpE, Fiu, MalPQ, oraA, nhoA, recA, otsAB and yciD.

72. The method of claim 67, wherein each of said first or said second analyte is independently selected from the group consisting of a condition and a substance.

73. The method of claim 67, wherein each of said at least two subpopulations of cells is tagged.

74. The method of claim 67, wherein analyzing expression is effected by a pattern recognition software.
